(19) United States
(12) Patent Application Publication
(10) Pub. No.: US 2023/0380760 A1
(43) Pub. Date: Nov. 30, 2023

(54) SYSTEMS AND METHODS FOR CLASSIFYING OPHTHALMIC DISEASE SEVERITY

(71) Applicant: Oregon Health & Science University, Portland, OR (US)

(72) Inventors: Yali Jia, Portland, OR (US); Pengxiao Zang, Portland, OR (US)

(73) Assignee: Oregon Health & Science University, Portland, OR (US)

(21) Appl. No.: 18/202,463

(22) Filed: May 26, 2023

Related U.S. Application Data
(60) Provisional application No. 63/346,721, filed on May 27, 2022.

(51) Int. Cl. A61B 5/00; A61B 3/10; A61B 3/12

(52) U.S. Cl. CPC A61B 5/4842; A61B 3/102; A61B 3/1241

(57) ABSTRACT

Methods and systems for identifying levels of an ophthalmic disease are described. An example method includes generating, by a convolutional neural network (CNN) and using a 3D image of a retina, a vector. The method further includes generating, by a first model and using the vector, a first likelihood that the retina exhibits a first level of an disease and generating, by a second model and using the vector, a second likelihood that the retina exhibits a second level of the disease. The method further includes determining whether the retina exhibits an absence of the ophthalmic disease, the first level of the disease, or the second level of the disease based on the first likelihood and the second likelihood. Further, an indication of whether the retina exhibits the absence of the ophthalmic disease, the first level of the ophthalmic disease, or the second level of the ophthalmic disease is output.

### CROSS-REFERENCE TO RELATED APPLICATION

This application claims the priority of U.S. Provisional App. No. 63/346,721, filed on May 27, 2022, and which is hereby incorporated by reference in its entirety.

### STATEMENT REGARDING FEDERALLY SPONSORED RESEARCH OR DEVELOPMENT

This invention was made with government support under R01 EY027833 and R01 EY024544 awarded by The National Institutes of Health. The government has certain rights in the invention.

### TECHNICAL FIELD

This disclosure relates generally to systems, devices, and methods for identifying and monitoring levels of diabetic retinopathy (DR) in subjects using noninvasive imaging techniques, such as optical coherence tomography (OCT) and/or optical coherence tomographic angiography (OCTA).

### BACKGROUND

Diabetic retinopathy (DR) is a leading cause of preventable blindness globally (Wilkinson C P et al., Ophthalmology, 2003; 110(9):1677-82). Currently, DR classification uses fundus photographs or clinical examination to identify referable DR (rDR) and vision-threatening DR (vtDR). Eyes with worse than mild nonproliferative DR (NPDR) on the International Diabetic Retinopathy Severity Scale are considered rDR, and eyes with severe NPDR, proliferative DR (PDR), or those with diabetic macular edema (DME) are considered vtDR (Wong T Y et al., Ophthalmology, 2018; 125(10): 1608-22). An efficient and reliable classification system is essential in identifying patients who can benefit from treatment without an undue burden to the clinic. Eyes with rDR but without vtDR can be observed closely without referral to an ophthalmologist, helping preserve scarce resources for patients that require treatment. To do this safely requires an accurate stratification of patients into these categories (Flaxel C J et al., Ophthalmology. 2020; 127(1):66-145; Antonetti D A et al., N. Engl. J. Med. 2012; 366:1227-39).

Deep learning has enabled multiple reliable automated systems that classify DR from fundus photographs (Gargeya R & Leng T, Ophthalmology. 2017; 124(7):962-69; Abramoff M D et al., Investig. Ophthalmol. Vis. Sci. 2016; 57(13):5200-06; Gulshan V, et al., JAMA. 2016; 316(22):2402-10; Ghosh R et al., Proc. 4th SPIN. 2017:550-54). However, fundus photographs have a low sensitivity (60-73%) and specificity (67-79%) for detecting diabetic macular edema (DME), which accounts for the majority of vision loss in DR (Lee R et al., Eye and vision. 2015; 2(1):1-25; Prescott G et al., Brit. J. Ophthalmol. 2014; 98(8):1042-49). This means that even when a network performs very well against a ground truth generated from fundus photographs, patients with DME may still frequently be misdiagnosed. Supplementing fundus photography with OCT, which is the current gold standard for diagnosing macular edema, can avoid this problem (Huang D et al., Science. 1991; 254(5035):1178-81; Virgili G et al., Cochrane Database Syst Rev. 2015; 1: CD008081; Kinyoun J et al., Ophthalmology. 1989; 96(6):746-50; Bhavsar K V & Subramanian M L, Br J Ophthalmol. 2011; 95(5):671-74; Bressler N M et al, Eye (Lond). 2012; 26(6):833-40; Browning D J & Fraser C M, Am J Ophthalmol. 2008; 145(1):149-54; Browning D J et al., Ophthalmology. 2008; 115(3):533-39; Ruia S et al., Asia Pac J Ophthalmol (Phila). 2016; 5(5):360-67; Olson J et al., Health Technol Assess. 2013; 17(51):1-142; Schmidt-Erfurth U et al., Ophthalmologica. 2017; 237(4):185-222). However, reliance on multiple imaging modalities is undesirable as it increases logistic challenges and cost.

Previous technologies have demonstrated that OCT angiography (OCTA) can stage DR according to fundus photography-derived DR severity scales using various biomarkers linked to capillary changes in DR (Makita S et al., Optics express. 2006; 14(17):7821-40; An L & Wang R K, Optics express, 2008; 16(15):11438-52; Jia Y et al., Opt. Express. 2012; 20(4):4710-25; Jia Y et al., Proc. Natl. Acad. Sci. 2015; 112(18):E2395-402; Hwang T S et al., JAMA ophthalmol. 2016; 134(12):1411-19; Zhang M et al., Investig. Ophthalmol. Vis. Sci. 2016; 57(13):5101-06; Hwang T S et al., JAMA ophthalmol. 2016; 134(4):367-73; Hwang T S et al., Retina. 2015; 35(11):2371). Because OCTA scans simultaneously acquire detailed structural images that can diagnose DME, an automated system based on OCTA volume scans can potentially use a single imaging modality to accurately classify DR while avoiding low DME detection sensitivities and associated misdiagnoses that occur in systems based on just fundus photographs.

Despite this advantage, OCTA-based analyses require improvements. Previous methods for classifying DR using OCTA relied on accurate retinal layer segmentation and en face visualization of the 3D volume to visualize or measure biomarkers (Sandhu H S et al., Investig. Ophthalmol. Vis. Sci. 2018; 59(7):3155-60; Sandhu H S et al., Brit. J. Ophthalmol. 2018; 102(11):1564-69; Alam M et al., Retina. 2020; 40(2):322-32; Heisler M et al., Transl. Vis. Sci. Technol. 2020; 9(2):20; Le D et al., Transl. Vis. Sci. Technol. 2020; 9(2):35; Zang P et al., IEEE transactions on Biomedical Engineering. 2021; 68(6):1859-70). However, with advanced pathology, retinal layer segmentation can become unreliable. This lowers OCTA yield rate and may also lead to misclassification through segmentation errors. In addition, quantifying only specific biomarkers fails to make use of the information in the latent feature space of the OCT/OCTA volumes, which may be helpful for DR classification (You Q S et al., JAMA Ophthalmol. 2021; 139(7):734-41).

### SUMMARY

Various implementations of the present disclosure relate techniques for accurately classifying DR using OCT and OCTA. In some cases, a single 3D volumetric image of a retina is obtained by performing OCT and OCTA imaging on the retina. In some examples, a two-dimensional (2D) en face image can be segmented and utilized instead of the single 3D volumetric image, using techniques similar to those described in Zang et al., IOVS. 2020; 61:1147 and Zang et al., IEEE Transactions on Biomedical Engineering. 2021; 68(6):1859-70. The 3D image is processed using a trained convolutional neural network (CNN) in order to yield a single-dimensional vector. The CNN, for instance, includes multiple convolution blocks that progressively reduce the dimensions of the 3D image into the single-dimensional vector. In some cases, the vector is generated without relying on any additional images beyond the single 3D image.

The vector is processed by multiple blocks in parallel, which are used to respectively calculate likelihoods that the retina exhibits different levels of an ophthalmic disease, such as DR. For example, a first block is used to determine a likelihood that the 3D image depicts a first level of the disease and a second block is used to determine a likelihood that the 3D image depicts a second level of disease, and so on. By comparing the likelihoods output from the parallel blocks, the level of disease depicted in the 3D image can be ascertained and output.

In various examples, the CNN is trained based on training data generated by expert graders. For instance, the expert graders review 7-field fundus images (and/or volumetric OCT images) of multiple retinas and indicate the levels of the ophthalmic disease that are exhibited by the retinas. Various parameters of the CNN are optimized based on the images and the indications of the levels of the ophthalmic disease.

According to some implementations, a CAM is generated. The CAM indicates one or more regions within the 3D image that are predicted to depict structures relevant to the level of the ophthalmic disease. The CAM may be displayed or otherwise indicated to a user.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The detailed description is described with reference to the accompanying figures. In the figures, the left-most digit(s) of a reference number identifies the figure in which the reference number first appears. The use of the same reference numbers in different figures indicates similar or identical components or features.

FIG. 1 illustrates an example environment for training and utilizing a predictive model to identify ophthalmic disease levels in subjects.

FIG. 2 illustrates an example of training data, which may be used to train a predictive model according to various implementations of the present disclosure.

FIG. 3 illustrates an example of a CNN.

FIG. 4 illustrates an example of a classifier.

FIG. 5 illustrates an example of a convolutional block in a neural network.

FIGS. 6A to 6C illustrate examples of dilation rates.

FIG. 7 illustrates an example process for training and utilizing a NN to determine a level of an ophthalmic disease exhibited by a subject.

FIG. 8 illustrates an example process for predicting a level of an ophthalmic disease exhibited by a subject.

FIG. 9 illustrates an example of one or more devices that can be used to implement any of the functionality described herein.

FIG. 10 illustrates an example automated DR classification framework using volumetric OCT and OCTA data as inputs.

FIG. 11 illustrates a detailed architecture of the novel 3D convolutional neural network (CNN). Sixteen convolutional blocks were used in this 3D CNN. Each convolutional block was constructed as 3D convolutional layer with batch normalization and ReLU activation. Five convolutional blocks with diminishing kernel size (5 to 3) were used to downsample the inputs.

FIG. 12 illustrates a detailed design of an example of the output layer. Two paratactic layers were used to detect referable DR (rDR) and vision threatening DR (vtDR), respectively. The class activation maps (CAMs) for rDR and vtDR were generated according to the weighted sum of the last feature map.

FIG. 13 illustrates an example of six en face OCT/OCTA images (for the 3D CAM evaluation) that were generated from 3D OCT and OCTA based on eight segmented retinal layer boundaries. The segmented boundaries are shown on the first B-frame of the 3D OCT. The eight boundaries from top to bottom are: Vitreous/ILM (red), NFL/GCL (green), IPL/INL (yellow), INL/OPL (indigo), OPL/ONL (magenta), ONL/EZ (red), EZ/RPE (cyan), and RPE/BM (blue). Three en face projections were generated from structural OCT: (A) Inner retinal (the slab between the Vitreous/inner limiting membrane and outer plexiform and outer nuclear layer boundaries) thickness map, (B) Inner retinal mean projection of the OCT reflectance, and (C) Ellipsoid zone (EZ) en face mean projection (Outer nuclear layer/ellipsoid zone boundary to ellipsoid zone/retinal pigment epithelium boundary). The other three en face maximum projections were generated from OCTA: (D) Maximum projection of the flow volume in the superficial vascular complex (SVC; inner 80% of the ganglion cell complex), (E) Intermediate capillary plexus (ICP; outer 20% of the ganglion cell complex and inner 50% of the inner nuclear layer), and (F) Deep capillary plexus (DCP; remaining slab internal to the outer boundary of the outer plexiform layer).

FIG. 14 illustrates the mean receiver operating characteristic (ROC) curve derived from the 5-fold cross-validation for rDR (right) and vtDR (left) classifications based on the example DR classification framework. The models achieve an AUC of 0.96±0.01 on rDR classification and AUC of 0.92±0.02 on vtDR classification.

FIG. 15 illustrates three confusion matrices for referable DR (rDR) classification, vision threatening DR (vtDR) classification, and multiclass DR classification based on the overall 5-fold cross-validation results.

FIG. 16 illustrates class activation maps (CAMs) based on the referable DR (rDR) output layer of the example framework for data from an eye with rDR without vision threatening DR (vtDR).

FIG. 17 illustrates class activation maps (CAMs) based on the vision threatening DR (vtDR) output layer of the example framework for data from an eye with vtDR but without DME.

FIG. 18 illustrates class activation maps (CAMs) based on vision threatening DR (vtDR) output layer of our framework for data from an eye with vtDR and DME.

FIG. 19 illustrates two-dimensional class activation maps (CAMs) generated by our previous study for data from an eye with vtDR and DME. Six en face projections covered with the same 2D CAMs are shown. The abnormal vessels and central macular fluid, which were highlighted regions in the 3D CAMs, were not weighted highly by the 2D CAM algorithm (red circles in the inner and EZ CAMs).

### DETAILED DESCRIPTION

This disclosure describes various techniques for classifying the severity of an ophthalmic disease of a retina. In particular systems, an OCT and/or OCTA image is obtained of the retina. For example, the image is a three-dimensional (3D) volumetric image of the retina. A system classifies the severity of an ophthalmic disease of the retina based on the image. In various cases the system stores and/or otherwise applies a trained neural network (e.g., a CNN), which the system uses to generate a vector based on the image of the retina. The system also includes a classifier that includes multiple blocks operating in parallel on the vector, wherein each block of the classifier is used to determine a likelihood that the retina has a particular level of the disease. Based on the likelihoods generated using the blocks, the system accurately classifies the level of disease depicted in the image.

Various implementations of the present disclosure are directed to technical improvements in the field of medical imaging, and more specifically, ophthalmic imaging. Previously, classification of ophthalmic diseases, such as DR, relied on manual evaluation of fundus images by a trained expert. This trained expert, in many cases, would be an ophthalmologist with specialized retina expertise. General practitioners may be unable to accurately identify the level of DR in fundus images. In low-resource settings without access to retina specialists, patients at risk of DR are unable to identify their DR disease level and are at risk of mismanaging their disease. The consequences of DR mismanagement can lead to dire consequences, like permanent blindness.

Implementations of the present disclosure address these and other problems by accurately classifying the level of DR (or other ophthalmic diseases) using OCT and OCTA. In particular cases, a trained neural network is used to classify the disease level with an accuracy comparable to trained retina specialists. Using various techniques described herein, clinicians in low-resource settings may nevertheless accurately track the ophthalmic disease progression of their patients, which can considerably improve patient care.

In some cases, a retina can be classified without relying on color fundus images of the retina. For instance, the level of an ophthalmic disease exhibited by the retina can be more accurately identified using an OCT/OCTA image as compared to fundus image-based techniques. Furthermore, the level can be accurately classified using a single image, rather than multiple (e.g., fundus) images, which provides enhanced accessibility and simplicity over fundus-based techniques.

Furthermore, techniques described in this disclosure can utilize a single image (e.g., an OCT/OCTA volumetric image) to accurately identify the disease level. By relying on a single imaging modality, various techniques described herein can accurately classify retinas with relatively few processing resources, as compared to techniques that require additional images and/or complex segmentation techniques.

### EXAMPLE DEFINITIONS

As used herein, the term “Optical Coherence Tomography (OCT),” and its equivalents, can refer to a noninvasive low-coherence interferometry technique that can be used to obtain depth images of tissues, such as structures within the eye. In various implementations, OCT can be used to obtain depth images of retinal structures (e.g., layers of the retina). In some cases, OCT can be used to obtain a volumetric image of a tissue. For example, by obtaining multiple depth images of retinal structures along different axes, OCT can be used to obtain a volumetric image of the retina.

As used herein, the term “Optical Coherence Tomographic Angiography (OCTA),” and its equivalents, can refer to a subset of OCT techniques that obtain images based on flow (e.g., blood flow) within an imaged tissue. Accordingly, OCTA can be used to obtain images of vasculature within tissues, such as the retina. In some cases, OCTA imaging can be performed by obtaining multiple OCT scans of the same area of tissue at different times, in order to analyze motion or flow in the tissue that occurred between the different times.

As used herein, the term “OCT image,” and its equivalents, can refer to an OCT reflectance image, an OCTA image, or a combination thereof. An OCT image may be two-dimensional (e.g., one 2D projection image or one 2D depth image) or three-dimensional (e.g., a volumetric image).

As used herein, the terms “vascular,” “perfusion,” and the like can refer to an area of an image that depicts vasculature. In some cases, a perfusion area can refer to an area that depicts a blood vessel or another type of vasculature.

As used herein, the terms “avascular,” “nonperfusion,” and the like can refer to an area of an image that does not depict vasculature. In some cases, a nonperfusion area can refer to an area between blood vessels or other types of vasculature.

As used herein, the terms “blocks,” “layers,” and the like can refer to devices, systems, and/or software instances (e.g., Application Programming Interfaces (APIs), Virtual Machine (VM) instances, or the like) that generates an output by apply an operation to an input. A “convolutional block,” for example, can refer to a block that applies a convolution operation to an input (e.g., an image). When a first block is in series with a second block, the first block may accept an input, generate an output by applying an operation to the input, and provide the output to the second block, wherein the second block accepts the output of the first block as its own input. When a first block is in parallel with a second block, the first block and the second block may each accept the same input and may generate respective outputs that can be provided to a third block. In some examples, a block may be composed of multiple blocks that are connected to each other in series and/or in parallel. In various implementations, one block may include multiple layers.

In some cases, a block can be composed of multiple neurons. As used herein, the term “neuron,” or the like, can refer to a device, system, and/or software instance (e.g., VM instance) in a block that applies a kernel to a portion of an input to the block.

As used herein, the term “kernel,” and its equivalents, can refer to a function, such as applying a filter, performed by a neuron on a portion of an input to a block.

As used herein, the term “pixel,” and its equivalents, can refer to at least one value that corresponds to an area or volume of an image. In a grayscale image, the value can correspond to a grayscale value of an area of the grayscale image. In a color image, the value can correspond to a color value of an area of the color image. In a binary image, the value can correspond to one of two levels (e.g., a 1 or a 0). The area or volume of the pixel may be significantly smaller than the area or volume of the image containing the pixel. In examples of a line defined in an image, a point on the line can be represented by one or more pixels. A “voxel” is an example of a pixel spatially defined in three dimensions.

As used herein, the terms “Rectified Linear Unit,” “ReLU,” and their equivalents, can refer to a layer and/or block configured to remove negative values (e.g., pixels) from an input image by setting the negative values to 0.

As used herein, the term “batch normalization,” and its equivalents, can refer to a layer and/or block configured to normalize input images by fixing activations to be zero-mean and with a unit standard deviation.

As used herein, the terms “softmax,” “softmax activation,” and their equivalents, can refer to a function that is a generalization of the logistic function for multiple dimensions.

As used herein, the terms “class activation map,” “CAM,” and their equivalents, can refer to a heatmap indicating the presence of one or more features in an image. For example, a CAM indicating features associated with DR depicted in an OCT and/or OCTA image may have the same pixel dimensions as the OCT and/or OCTA image, wherein a value of each pixel in the CAM indicates a probability that the corresponding pixel in the OCT and/or OCTA image depicts a feature associated with DR.

### Particular Implementations

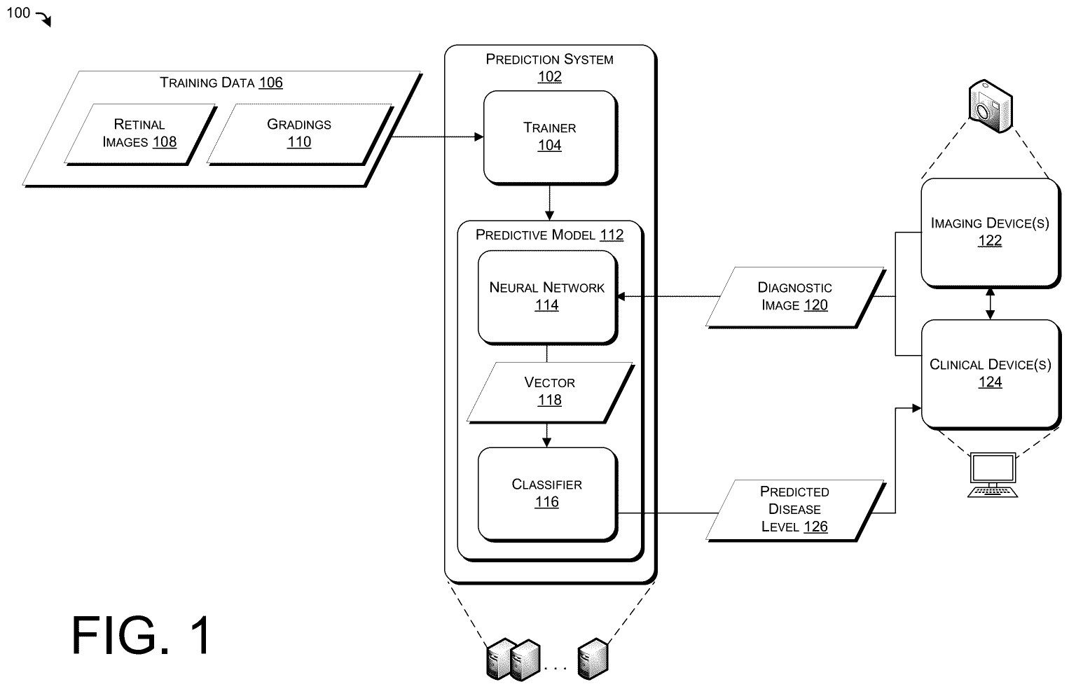

Some particular implementations of the present disclosure will now be described with reference to FIGS. 1-19. However, the implementations described with reference to FIGS. 1-19 are not exhaustive.

FIG. 1 illustrates an example environment 100 for training and utilizing a predictive model to identify ophthalmic disease levels in subjects. As shown in FIG. 1, the environment 100 includes a prediction system 102, which may be configured to identify an ophthalmic disease level in various example subjects. The prediction system 102, for example, is embodied in one or more computing devices (e.g., servers). The prediction system 102 may include hardware, software, or a combination thereof.

The prediction system 102 may include a trainer 104, which can receive training data 106. The trainer 104 may use the training data 106 to train one or more models to identify ophthalmic disease levels in subjects. In various implementations, the training data 106 can include previously obtained retinal images 108 of various individuals in a sample population. For example, these retinal images 108 may include OCT-based images.

In various implementations, the retinal images 108 are volumetric images that depict the retinas of the various individuals. According to some examples, an individual volumetric image includes multiple voxels respectively corresponding to volumes within an example retina of the various individuals. An example voxel has at least one value corresponding to the corresponding example volume. In various implementations, the example volume has one value corresponding to the OCT value of the example volume and a second value corresponding to the OCTA value of the example volume. The retinal images 108, for example, are generated by one or more combination OCT/OCTA scanners. In some cases, the retinal images 108 may be obtained by obtaining multiple OCT and OCTA depth scans of each of the retinas at various axes. According to some instances, the retinal images 108 within the training data 106 include a single image per retina of the various individuals in the sample population. In various implementations, macular edema is discernible in the retinal images 108. For example, fundus images are omitted from the training data 106.

According to various implementations, the retinal images 108 depict a variety of different retinas. For example, the retinal images 108 may depict retinas with the ophthalmic disease and retinas without the ophthalmic disease. The retinal images 108, in various cases, depict retinas with a first level of the ophthalmic disease, a second level of the ophthalmic disease, and/or an nth level of the ophthalmic disease, wherein n is a positive integer greater than one. In examples wherein the ophthalmic disease is DR, for instance, the retinal images 108 depict retinas without DR, retinas with rDR, and retinas with vtDR.

In some implementations, the training data 106 further includes gradings 110 associated with the retinal images. The gradings 110 may be generated by one or more expert graders (e.g., retina specialists) who have identified the ophthalmic disease levels of the retinas depicted in the retinal images 108. For example, one or more retina specialists may have reviewed the retinal images 108 in the training data 106, other images of the retinas depicted in the training data 106 (e.g., fundus images), or have otherwise examined the retinas of the various individuals for disease progression.

In various examples, the trainer 104 is configured to use the training data 106 to train a predictive model 112, which includes a neural network 114 and a classifier 116. In some cases, the predictive model 112 is a deep learning model, such as a Convolutional Neural Network (CNN) model. For instance, the neural network 114 may include at least one CNN. The neural network 114 may be configured to generate a vector 118 that is input into the classifier 116 for further processing. The vector 118, for example, is data that has at least one dimension that is smaller than the corresponding dimension of each of the retinal images 108.

The term “Neural Network (NN),” and its equivalents, may refer to a model with multiple hidden layers, wherein the model receives an input (e.g., an image) and transforms the input by performing operations via the hidden layers. An individual hidden layer may include multiple “neurons,” each of which may be disconnected from other neurons in the layer. An individual neuron within a particular layer may be connected to multiple (e.g., all) of the neurons in the previous layer. A NN may further include at least one fully connected layer that receives a feature map output by the hidden layers and transforms the feature map into the output of the NN.

As used herein, the term “CNN,” and its equivalents, may refer to a type of NN model that performs at least one convolution (or cross correlation) operation on an input image and may generate an output image based on the convolved (or cross-correlated) input image. A CNN may include multiple layers that transforms an input image (e.g., a 3D volume) into an output image via a convolutional or cross-correlative model defined according to one or more parameters. The parameters of a given layer may correspond to one or more filters, which may be digital image filters that can be represented as images. A filter in a layer may correspond to a neuron in the layer. A layer in the CNN may convolve or cross correlate its corresponding filter(s) with the input image in order to generate the output image. In various examples, a neuron in a layer of the CNN may be connected to a subset of neurons in a previous layer of the CNN, such that the neuron may receive an input from the subset of neurons in the previous layer and may output at least a portion of an output image by performing an operation (e.g., a dot product, convolution, cross-correlation, or the like) on the input from the subset of neurons in the previous layer. The subset of neurons in the previous layer may be defined according to a “receptive field” of the neuron, which may also correspond to the filter size of the neuron. U-net (see, e.g., Ronneberger, et al., arXiv:1505.04597v1, 2015) is an example of a CNN model.

The retinal images 108 represent inputs for the predictive model 112, and the gradings 110 represent outputs for the predictive model 112. The trainer 104 can perform various techniques to train (e.g., optimize the parameters of) the neural network 114 and/or the classifier 116 using the training data 106. For instance, the trainer 104 may perform a training technique utilizing stochastic gradient descent with backpropagation, or any other machine learning training technique known to those of skill in the art. In some implementations, the trainer 104 utilizes adaptive label smoothing to reduce overfitting. According to some cases, the trainer 104 applies L1-L2 regularization and/or learning rate decay to train the neural network 114 and/or classifier 116.

In various implementations, the trainer 104 may be configured to train the predictive model 112 by optimizing various parameters within the predictive model 112 based on the training data 106. For example, the trainer 104 may input the retinal images 108 into the predictive model 112 and compare outputs of the predictive model 112 to the gradings 110. The trainer 104 may further modify various parameters of the predictive model 112 (e.g., filters in the neural network 114) in order to ensure that the outputs of the predictive model 112 are sufficiently similar and/or identical to the gradings 110. For instance, the trainer 104 may identify values of the parameters that result in a minimum of loss between the outputs of the predictive model 112 and the gradings 110.

By optimizing the parameters of the predictive model 112, the trainer 104 may train the predictive model 112 to identify the level of the ophthalmic disease in a diagnostic image 120. The diagnostic image 120 is obtained by at least one imaging device 122 and/or at least one clinical device 124. The imaging device(s) 122 may include, for example, an OCT and/or OCTA imaging device. In some cases, the imaging device(s) 122 may include at least one camera, which may generate digital images (e.g., 3D volumetric images) of the retina of a subject based on a combined OCT and OCTA scan. In some cases, the imaging device 122 further obtains at least some of the retinal images 108 in the training data 106. Accordingly, in some implementations, the retinal images 108 and the diagnostic image 120 are generated using the same imaging system. In some cases, the retinal images 108 and the diagnostic image 120 are generated using the same type of imaging system, such as the same model of imaging system produced by the same manufacturer.

In various examples, the imaging device(s) 122 are a single imaging device that is configured to perform OCT and OCTA imaging. The imaging device(s) 122 may generate the diagnostic image 120 noninvasively (e.g., without requiring the use of contrast agents administered to the subject). In some implementations, the imaging device(s) 122 are located outside of a clinical environment. For example, the imaging device(s) 122 may be an at-home OCT/OCTA imaging device that can be operated by the subject. In some cases, the imaging device(s) 122 transmit the diagnostic image 120 to an external device. For instance, the prediction system 102 and/or clinical device(s) 124 may be located remotely from the imaging device(s) 122.

In a particular example, the imaging device(s) 122 obtains the diagnostic image 120 by performing a combination OCT/OCTA scan on a subject. The diagnostic image 120 may include 3D volumetric image of a retina of the subject. For example, the diagnostic image 120 may include various voxels, wherein an individual voxel includes a first value corresponding to the OCT level at an example volume in the field-of-view of the imaging device(s) 122 and a second value corresponding to the OCTA level at the example volume. In cases where the diagnostic image 120 is a 3D volumetric image, the diagnostic image 120 omits a projection (e.g., en face) image of the retina. Furthermore, in these and other cases, segmentation of different layers of the retina is unnecessary to generate the diagnostic image 120.

The imaging device 122 can provide the diagnostic image 120 to the prediction system 102 executing the predictive model 112. The predictive model 112 may have been previously trained by the trainer 104. The neural network 114 may generate a vector 118 based on the diagnostic image 120. For example, the neural network 114 may be used to perform one or more convolutions and/or cross-correlations on the diagnostic image 120. According to various implementations, the neural network 114 may include multiple convolution blocks, arranged in series. The series of convolution blocks may downsample the diagnostic image 120. For instance, the series of convolution blocks may have diminishing kernel sizes. According to various implementations, the trainer 104 may have optimized various filters and/or other parameters within the series of convolution blocks based on the training data 106. In various cases, the diagnostic image 120 may have dimensions of x by y by z voxels and the vector 118 may have dimensions of a by b by c data, where at least one of a<x, b<y, or c<z. In some cases, the vector 118 is a one-dimensional set of data.

The classifier 116 may include multiple blocks arranged in parallel. An individual block within the classifier 116 may be used to generate a likelihood that the diagnostic image 120 depicts a particular level of the ophthalmic disease. Accordingly, the classifier 116 is used to generate multiple likelihoods, respectively corresponding to different levels of the ophthalmic disease. In cases where the ophthalmic disease is DR, one block may be used to generate a likelihood that the diagnostic image 120 depicts rDR and another block may be used to generate a likelihood that the diagnostic image 120 depicts vtDR. The classifier 116 may include blocks that evaluate the likelihood the diagnostic image 120 depicts other levels of DR. In some cases, the ophthalmic disease is age-related macular degeneration (AMD) and/or glaucoma. The classifier 116 may determine likelihoods that the diagnostic image 120 depicts different severity levels of the ophthalmic disease. In various implementations, a predicted disease level 126 of the retina depicted by the diagnostic image 120 is generated based on the likelihoods generated by the classifier 116.

The prediction system 102 executing the predicative model 112 may output the predicted disease level 126 to the clinical device(s) 124. In various implementations, the clinical device(s) 124 may output the predicted disease level 126 to a user (e.g., a clinician) via a user interface. For example, the clinical device(s) 124 may output the predicted disease level 126 on a display of the clinical device(s) 124 or audibly by a speaker.

Although not specifically illustrated in FIG. 1, in various implementations, the prediction system 102 is further configured to generate and/or output a CAM based on the diagnostic image 120. The CAM is an image representing one or more disease regions in the diagnostic image 120. The disease region(s) correspond to structures within the retina that are indicative of the level of the ophthalmic disease of interest. For example, if the diagnostic image 120 depicts a retina with DR, the CAM may indicate a region of macular edema within the diagnostic image 120. The CAM, for instance, may be a heatmap that highlights the disease region(s).

In various implementations, the classifier 116 is used to generate the CAM based on the vector 118 and/or the diagnostic image 120. The CAM may be provided to the clinical device(s) 124 and may be output to a user by the clinical device(s) 124. For instance, the clinical device(s) 124 may display the CAM on a screen. Accordingly, the user may confirm the predicted disease level 126 by manually observing the disease level-relevant region(s) identified by the prediction system 102.

In some implementations, the prediction system 102 may be hosted on one or more devices (e.g., servers) that are located remotely from the clinical device(s) 124. For example, the prediction system 102 may receive and evaluate diagnostic images from multiple imaging devices and/or clinical devices located in various locations (e.g., various healthcare facilities).

According to certain implementations, the prediction system 102 and/or the clinical device(s) 124 may interface with an Electronic Medical Record (EMR) system (not illustrated). The diagnostic image 120, the predicted disease level 126, and the like, may be stored and/or accessed in memory stored at the EMR system.

In various implementations, at least one of the prediction system 102, the predictive model 112, the imaging device(s) 122, or the clinical device(s) 124 may include at least one system (e.g., a distributed server system), at least one computing device, at least one software instance (e.g., a VM) hosted on system(s) and/or device(s), or the like. For instance, instructions to execute functions associated with at least one of prediction system 102, the predictive model 112, the imaging device(s) 122, or the clinical device(s) 124 may be stored in memory. The instructions may be executed, in some cases, by at least one processor.

According to various examples, at least one of the training data 106, the diagnostic image 120, the vector 118, or the predicted disease level 126 may include data packaged into at least one data packet. In some examples, the data packet(s) can be transmitted over wired and/or wireless interfaces. According to some examples, the data packet(s) can be encoded with one or more keys stored by at least one of the prediction system 102, the trainer 104, the predictive model 112, the imaging device(s) 122, or the clinical device(s) 124, which can protect the data paged into the data packet(s) from being intercepted and interpreted by unauthorized parties. For instance, the data packet(s) can be encoded to comply with Health Insurance Portability and Accountability Act (HIPAA) privacy requirements. In some cases, the data packet(s) can be encoded with error-correcting codes to prevent data loss during transmission.

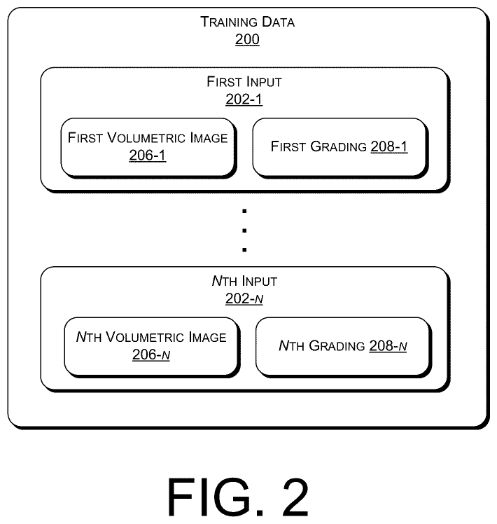

FIG. 2 illustrates an example of training data 200, which may be used to train a predictive model according to various implementations of the present disclosure. In some cases, the training data 200 can be and/or include the training data 106 described above with reference to FIG. 1.

The training data 200 may include n inputs 202-1 to 202-n, wherein n is a positive integer. The inputs 202-1 to 202-n may respectively include volumetric images 206-1 to 206-n and gradings 208-1 to 208-n. Each one of the inputs 202-1 to 202-n may correspond to a retina of a single individual obtained at a particular time. For example, a first input 202-1 may include a first volumetric image 206-1 of a first retina of a first example individual that was scanned on a first date, and a second input may include a volumetric image of a second retina of a second example individual that was scanned on a second date. In some cases, the first individual and the second individual can be the same person, but the first date and the second date may be different days. In some implementations, the first individual and the second individual can be different people, but the first date and the second date can be the same days.

The first to nth gradings 208-1 to 208-n may indicate the level of an ophthalmic disease depicted in the first to nth volumetric images 206-1 to 206-n, respectively. In various cases, the first to nth gradings 208-1 to 208-n are generated by one or more experts, such as one or more retina specialists. In some cases, the expert(s) rely on different images than the first to nth volumetric images 206-1 to 206-n in order to generate the gradings 208-1 to 208-n. For instance, the volumetric images 206-1 to 206-n may be OCT and/or OCTA images, but the expert(s) may generate the gradings 208-1 to 208-n based on fundus images.

According to various implementations, the training data 200 is used to train a predictive model. In some examples, the predictive model includes at least one CNN including various parameters that are optimized based on the training data 200. For instance, the training data 200 may be used to train a CNN configured to generate a vector that is used to classify the level of an ophthalmic disease depicted in a diagnostic image of a subject's retina.

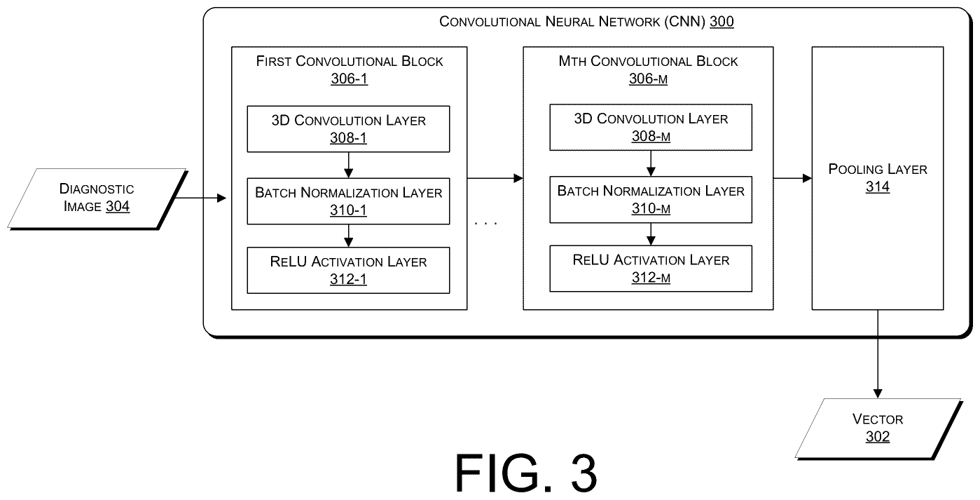

FIG. 3 illustrates an example of a CNN 300, which may be included in the neural network 114 described above with reference to FIG. 1. As illustrated, the CNN 300 includes multiple blocks that generate a vector 302 based on a diagnostic image 302.

The CNN 300 includes first to mth convolutional blocks 306-1 to 306-m, wherein m is a positive integer. The convolutional blocks 306-1 to 306-m are arranged in series. In various implementations, the first to mth convolutional blocks 306-1 to 306-m include first to mth 3D convolution layers 308-1 to 308-m, first to mth batch normalization layers 310-1 to 310-m, and first to mth ReLU activation layers 312-1 to 312-m. For instance, the first convolutional block 306-1 includes a first convolution layer 308-1, a first batch normalization layer 310-1, and a first ReLU activation layer 312-1, which are arranged in series.

In various implementations, the diagnostic image 304 is processed by the series of first to mth convolutional blocks 306-1 to 306-m. In some cases, the diagnostic image 304 is resized prior to being input into the first convolutional block 306-1. Each of the convolutional blocks 306-1 to 306-m (e.g., each of the convolution layers 308-1 to 308-m in the convolutional blocks 306-1 to 306-m) may be defined according to a kernel size, an output channel, and a stride size. In various implementations, the kernel of a convolutional block 306-1 to 306-m (e.g., of a convolution layer 308-1 to 308-m) is defined in three dimensions. The kernel (or “filter”) of a given convolution block or layer is convolved and/or cross-correlated with an input to the block or layer. A 3D kernel can be represented as a 3D matrix, in some implementations. The kernels of the convolutional blocks 306-1 to 306-m may be cubic, such that the length, width, and height of a given kernel is defined by the same size. For instance, the kernel size in the convolutional blocks 306-1 to 306-m may be 2×2×2, 3×3×3, 4×4×4, 5×5×5, 6×6×6, or the like. In some cases, the convolutional blocks 306-1 to 306-m may utilize kernels of different sizes. During training, the values of the kernels may be optimized based on the training data.

The stride size of a convolutional block 306-1 to 306-m (e.g., of a convolution layer 308-1 to 308-m) corresponds to the distance between pixels that are convolved and/or cross-correlated with the kernel at a given time. A stride size of 1 indicates that the kernel is convolved and/or cross-correlated with adjacent pixels in the input. A stride size of 2 indicates that the kernel is convolved and/or cross-correlated with pixels in the input that are spaced apart by one pixel. In various implementations, the convolutional blocks 306-1 to 306-m have strides of 1, 2, 3, or the like. In some cases, the convolutional blocks 306-1 to 306-m may utilize strides of different sizes.

The batch normalization layers 310-1 to 310-m may be configured to mitigate and/or correct a covariate shift that occurs due to the convolution layers 308-1 to 308-m. The inclusion of the batch normalization layers 310-1 to 310-m may increase training efficiency. Examples of batch normalization are described, for instance, in Ioffe et al., arXiv:1502.03167 [cs.LG] (2015).

The ReLU layers 312-1 to 312-m may be configured to receive an input that may include negative, positive, and zero values and may output positive and zero values. For example, the ReLU layers 312-1 to 312-m may convert a negative input value into a zero-output value.

The output of the mth convolution block 306-m is processed via a pooling layer 314 within the CNN 300. The pooling layer 314 is used to generate the vector 302. In various implementations, the pooling layer 314 applies an average pooling and/or maximum pooling function to the output of the mth convolutional block 306-m in order to generate the vector 302.

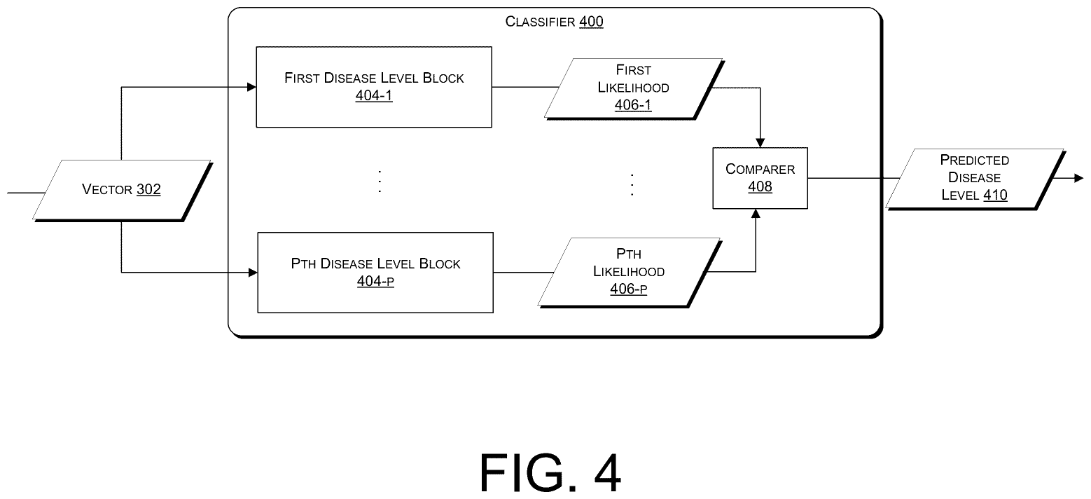

FIG. 4 illustrates an example of a classifier 400. In some implementations, the classifier 400 is the classifier 116 described above with respect to FIG. 1.

The classifier 400 includes first to pth disease level blocks 404-1 to 404-p, wherein p is an integer greater than one. Each individual disease level blocks 404-1 to 404-p is configured to predict whether the diagnostic image 304 depicts a particular level of an ophthalmic disease. That is, the first to pth disease level blocks 404-1 to 404-p are configured to respectively generate first to pth likelihoods 406-1 to 406-p. In various implementations, the first to pth disease level blocks 404-1 to 404-p include a ReLU layer, a probability layer, a softmax layer, or any combination thereof. The ReLU layer, for example, may perform a function in which the vector 302 is multiplied by a ReLU of a set of weight parameters. The weight parameters, for example, are optimized during training. The probability layer, according to various implementations, includes processing the output of the ReLU layer with one or more additional parameters. The additional parameter(s) may be optimized during training. The vector 302 is input into each one of the first to pth disease level blocks 404-1 to 404-p.

In particular examples, each of the first to pth disease level blocks 404-1 to 404-p includes at least three layers. For instance, the first disease level block 404-1 includes a first layer that is used to generate a first intermediary matrix by multiplying the vector by a ReLU of weight parameters associated with the first level of the ophthalmic disease; a second layer that is used to generate a first probability matrix based on the first intermediary matrix and first parameters; and a third layer used to generate the first likelihood by performing softmax activation on the first probability matrix.

In various implementations, the classifier 400 is used to determine the level of DR depicted in a retina depicted in the diagnostic image 304. For instance, the first disease level block 404-1 may be configured to determine the first likelihood 406-1, which may represent the likelihood that the retina exhibits rDR. A second disease level block 404-2 may be configured to determine a second likelihood representing the likelihood that the retina exhibits vtDR.

In various implementations, a comparer 408 is used to determine a predicted disease level 410 based on the first to pth likelihoods 406-1 to 406-p. The comparer 408 may compare the first to pth likelihoods 406-1 to 406-p. In some implementations, the comparer 408 determines the greatest likelihood among the first to pth likelihoods 406-1 to 406-p and defines the predicted disease level 410 as the level evaluated by the disease level block that produced the greatest likelihood. In some examples wherein at least one of the first to pth likelihoods 406-1 to 406-p is below at least one threshold, the comparer 408 may conclude that the disease is not present in the retina. In some cases, the comparer 408 generates the predicted disease level 410 to indicate that the retina is not predicted to have the ophthalmic disease if one or more if the first to pth likelihoods 406-1 to 406-p are below one or more thresholds.

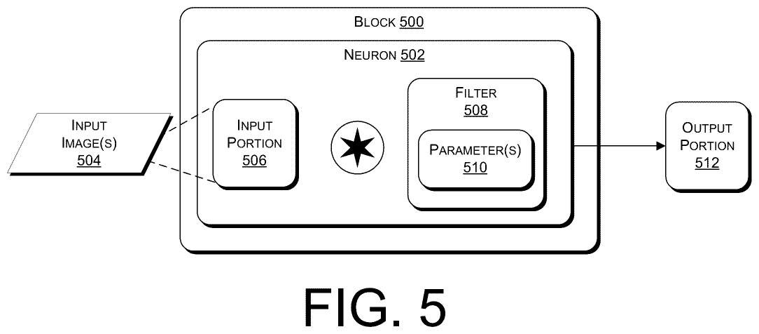

FIG. 5 illustrates an example of a convolutional block 500 in a neural network. In some examples, the block 500 can represent any of the convolutional blocks and/or layers described herein.

The convolutional block 500 may include multiple neurons, such as neuron 502. In some cases, the number of neurons may correspond to the number of pixels in at least one input image 504 input into the block 500. Although one neuron is illustrated in each of FIG. 5, in various implementations, block 500 can include multiple rows and columns of neurons.

In particular examples, the number of neurons in the block 500 may be less than or equal to the number of pixels in the input image(s) 504. In some cases, the number of neurons in the block 500 may correspond to a “stride” of neurons in the block 500. In some examples in which first and second neurons are neighbors in the block 500, the stride may refer to a lateral difference in an input of the first neuron and an input of the second neuron. For example, a stride of one pixel may indicate that the lateral difference, in the input image(s) 504, of the input of the first neuron and the input of the second neuron is one pixel.

Neuron 502 may accept an input portion 506. The input portion 506 may include one or more pixels in the input image(s) 504. A size of the input portion 506 may correspond to a receptive field of the neuron 502. For example, if the receptive field of the neuron 502 is a 3×3 pixel area, the input portion 506 may include at least one pixel in a 3×3 pixel area of the input image(s) 504. The number of pixels in the receptive field that are included in the input portion 506 may depend on a dilation rate of the neuron 502.

In various implementations, the neuron 502 may convolve (or cross-correlate) the input portion 506 with a filter 508. The filter may correspond to at least one parameter 510, which may represent various optimized numbers and/or values associated with the neuron 502. In some examples, the parameter(s) 610 are set during training of a neural network including the block 600.

The result of the convolution (or cross-correlation) performed by the neuron 502 may be output as an output portion 512. In some cases, the output portion 512 of the neuron 502 is further combined with outputs of other neurons in the block 500. The combination of the outputs may, in some cases, correspond to an output of the block 500. Although FIG. 5 depicts a single neuron 502, in various examples described herein, the block 500 may include a plurality of neurons performing operations similar to the neuron 502. In addition, although the convolutional block 500 in FIG. 5 is depicted in two dimensions, in various implementations described herein, the convolutional block 500 may operate in three dimensions.

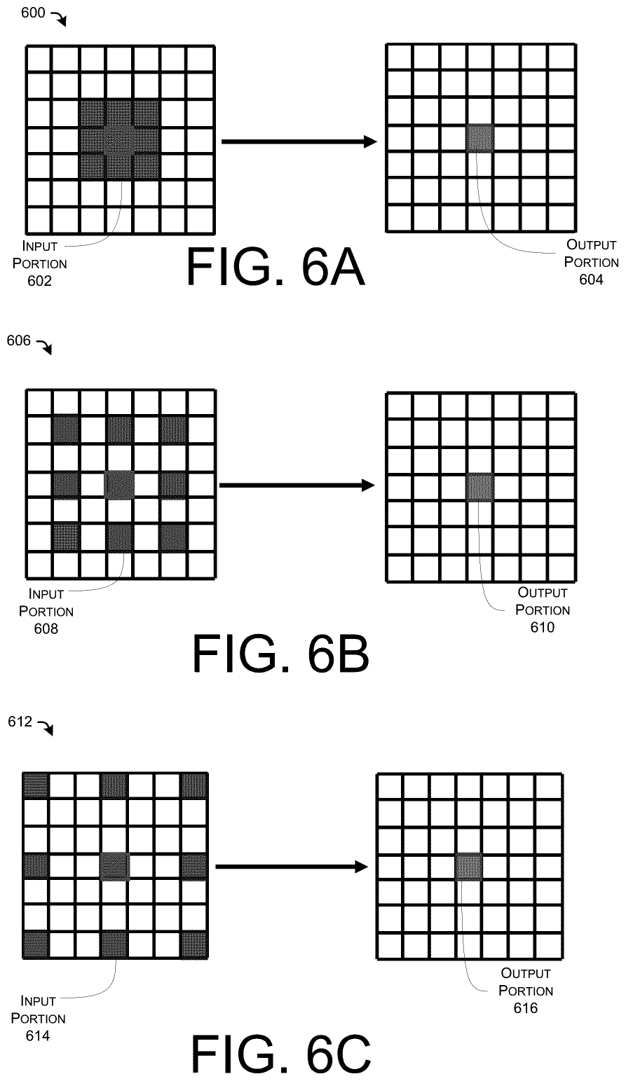

FIGS. 6A to 6C illustrate examples of dilation rates. In various implementations, the dilation rates illustrated in FIGS. 6A to 6C can be utilized by a neuron, such as the neuron 502 illustrated in FIG. 5. Although FIGS. 6A to 6C illustrate 2D dilation rates (with 3×3 input pixels and 1×1 output pixel), implementations can apply 3D dilation rates (with 3×3×3 input pixels and 1×1 output pixel).

FIG. 6A illustrates a transformation 600 of a 3×3 pixel input portion 602 into a 1×1 pixel output portion 604. The dilation rate of the transformation 600 is equal to 1. The receptive field of a neuron utilizing the transformation 600 is a 3×3 pixel area.

FIG. 6B illustrates a transformation 606 of a 3×3 pixel input portion 608 into a 1×1 pixel output portion 610. The dilation rate of the transformation 606 is equal to 2. The receptive field of a neuron utilizing the transformation 606 is a 5×5 pixel area.

FIG. 6C illustrates a transformation 612 of a 3×3 pixel input portion 614 into a 1×1 pixel output portion 616. The dilation rate of the transformation 612 is equal to 4. The receptive field of a neuron utilizing the transformation 600 is a 9×9 pixel area.

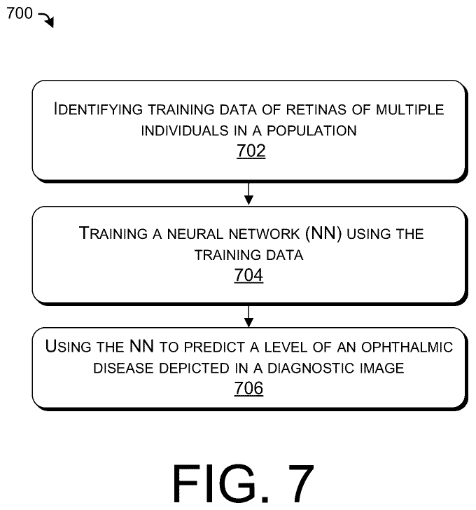

FIG. 7 illustrates an example process 700 for training and utilizing a NN to determine a level of an ophthalmic disease exhibited by a subject. The process 700 may be performed by an entity, such as the prediction system 102, a computing system, a processor, or any combination thereof.

At 702, the entity identifies training data of retinas of multiple individuals in a population. The training data, for example, includes OCT and/or OCTA images of retinas of the individuals. In some cases, the images include 3D volumetric images of the retinas. According to some implementations, the training data further indicates levels of an ophthalmic disease that are depicted by the images. For instance, the training data indicates that one image depicts vtDR and another image depicts rDR. In some cases, the images depict at least one retina without the ophthalmic disease. In some instances, the images depict at least one retina with each one of the levels of the ophthalmic disease.

At 704, the entity trains an NN using the training data. The NN may include various parameters that are optimized based on the training data. For example, the parameters are modified such that the outputs of the NN (when the images are inputs of the NN) are the levels of the training data with a minimum of loss.

At 706, the entity uses the NN to predict a level of an ophthalmic disease depicted in a diagnostic image. According to various implementations, an additional retinal image is input into the trained NN. The output is a predicted level of the ophthalmic disease that is depicted in the additional retinal image. In various implementations, the additional retinal image depicts the retina of an individual that is not part of the population used to generate the training data. In some cases, the entity further outputs a CAM indicative of one or more features in the additional retinal image that are relevant to the predicted level of the ophthalmic disease.

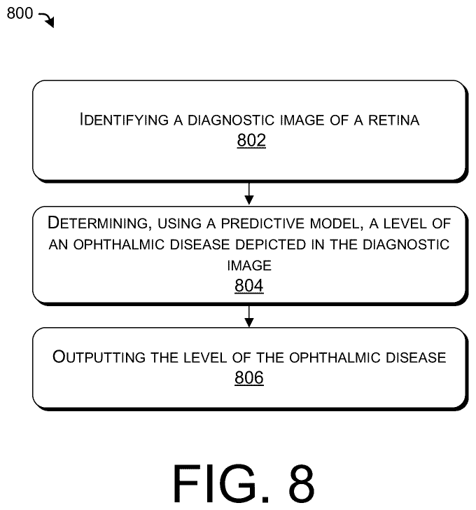

FIG. 8 illustrates an example process 800 for predicting a level of an ophthalmic disease exhibited by a subject. The process 800 may be performed by an entity, such as the prediction system 102, a computing system, a processor, or any combination thereof.

At 802, the entity identifies a diagnostic image of a retina. In various implementations, the diagnostic image is an OCT and/or OCTA image. According to some cases, the diagnostic image is a 3D volumetric image. For example, an example voxel of the diagnostic image includes a value corresponding to the OCT level of a volume of the retina and another value corresponding to the OCTA level of the volume of the retina.

At 804, the entity determines, using a predictive model, a level of an ophthalmic disease depicted in the diagnostic image. In some cases, the predictive model includes a trained NN, such as a CNN. In various implementations, the NN outputs a vector. The vector may be input into multiple parallel disease level blocks, that respectively output predicted likelihoods that the retina has different levels of an ophthalmic disease. A comparer may be used to determine which of the levels is the predicted level of the ophthalmic disease.

At 806, the entity outputs the level of the ophthalmic disease. In some cases, the entity causes the level to be visually output on a screen. In some cases, the entity generates a CAM indicative of one or more features in the image that are relevant to the predicted level of the ophthalmic disease. The entity, for example, generates the CAM based on the vector. The entity may cause the CAM to further be visually output on the screen.

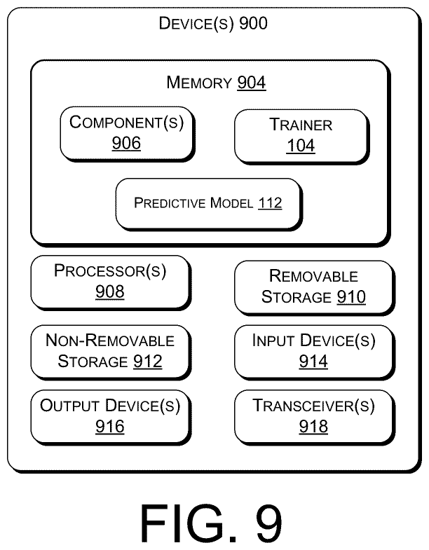

FIG. 9 illustrates an example of one or more devices 900 that can be used to implement any of the functionality described herein. In some implementations, some or all of the functionality discussed in connection with FIGS. 1-8 can be implemented in the device(s) 900. Further, the device(s) 900 can be implemented as one or more server computers, a network element on a dedicated hardware, as a software instance running on a dedicated hardware, or as a virtualized function instantiated on an appropriate platform, such as a cloud infrastructure, and the like. It is to be understood in the context of this disclosure that the device(s) 900 can be implemented as a single device or as a plurality of devices with components and data distributed among them.

As illustrated, the device(s) 900 include a memory 904. In various embodiments, the memory 904 is volatile (such as RAM), non-volatile (such as ROM, flash memory, etc.) or some combination of the two.

The memory 904 may store, or otherwise include, various components 906. In some cases, the components 906 can include objects, modules, and/or instructions to perform various functions disclosed herein. The components 906 can include methods, threads, processes, applications, or any other sort of executable instructions. The components 906 can include files and databases. For instance, the memory 904 may store instructions for performing operations of any of the trainer 104 or the predictive model 112.

In some implementations, at least some of the components 906 can be executed by processor(s) 908 to perform operations. In some embodiments, the processor(s) 908 includes a Central Processing Unit (CPU), a Graphics Processing Unit (GPU), or both CPU and GPU, or other processing unit or component known in the art.

The device(s) 900 can also include additional data storage devices (removable and/or non-removable) such as, for example, magnetic disks, optical disks, or tape. Such additional storage is illustrated in FIG. 900 by removable storage 910 and non-removable storage 912. Tangible computer-readable media can include volatile and nonvolatile, removable and non-removable media implemented in any method or technology for storage of information, such as computer readable instructions, data structures, program modules, or other data. The memory 904, removable storage 910, and non-removable storage 912 are all examples of computer-readable storage media. Computer-readable storage media include RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information, and which can be accessed by the device(s) 900. Any such tangible computer-readable media can be part of the device(s) 900.

The device(s) 900 also can include input device(s) 914, such as a keypad, a cursor control, a touch-sensitive display, voice input device, etc., and output device(s) 916 such as a display, speakers, printers, etc. In some implementations, the input device(s) 914, in some cases, may include a device configured to capture OCT images, such as OCT and/or OCTA images. In certain examples, the output device(s) 916 can include a display (e.g., a screen, a hologram display, etc.).

As illustrated in FIG. 9, the device(s) 900 can also include one or more wired or wireless transceiver(s) 916. For example, the transceiver(s) 916 can include a Network Interface Card (NIC), a network adapter, a Local Area Network (LAN) adapter, or a physical, virtual, or logical address to connect to the various base stations or networks contemplated herein, for example, or the various user devices and servers. The transceiver(s) 916 can include any sort of wireless transceivers capable of engaging in wireless, Radio Frequency (RF) communication. The transceiver(s) 916 can also include other wireless modems, such as a modem for engaging in Wi-Fi, WiMAX, Bluetooth, or infrared communication.

### Example: A Diabetic Retinopathy Diagnosis Framework Based on Deep-Learning Analysis of OCT Angiography

This example provides an automated convolutional neural network (CNN) that uses the whole (unsegmented) OCT/OCTA volume to directly classify eyes as either non-rDR (nrDR) or rDR, and as vtDR or non-vtDR (nvtDR) (LeCun Y et al., Nature. 2015; 521(7553):436-44). The example also includes a multiclass classification that classifies eyes as nrDR, rDR/nvtDR, (eyes with referable but not vision-threatening DR) or vtDR. To demonstrate which features the framework relies on to make the classification, the network also generates 3D class activation maps (CAMs) (Zhou B et al., Proceedings of the IEEE Computer Society Conference on Computer Vision and Pattern Recognition. 2016:2921-29). Visualizations such as these can be used as part of direct classification systems, since they allow graders to verify algorithm outputs. This example provides a unique automated multiclass DR severity-level classification framework based directly on OCT and OCTA volumes.

### Methods

### Data Acquisition

50 healthy participants and 305 patients with diabetes were recruited and examined at the Casey Eye Institute, Oregon Health & Science University in the United States (50 healthy participants and 234 patients); Shanxi Eye Hospital in China (60 patients); and the Department of Ophthalmology, Aichi Medical University in Japan (11 patients). Diabetic patients were included with the full spectrum of disease from no clinically evident retinopathy to proliferative diabetic retinopathy. One or both eyes of each participant underwent 7-field color fundus photography and an OCTA scan using a commercial 70-kHz spectral-domain OCT (SD-OCT) system (RTVue-XR Avanti, Optovue Inc) with 840-nm central wavelength. The scan depth was 1.6 mm in a 3.0×3.0 mm region (640×304×304 pixels) centered on the fovea. Two repeated B-frames were captured at each line-scan location. The structural images were obtained by averaging the two repeated and registered B-frames. Blood flow was detected using the split-spectrum amplitude-decorrelation angiography (SSADA) algorithm (Jia Y et al., Opt. Express. 2012; 20(4):4710-25; Gao S S et al., Opt. Lett. 2015; 40(10):2305-08). For each volumetric OCT/OCTA, two continuously acquired volumetric raster scans (one x-fast scan and one y-fast scan) were registered and merged through an orthogonal registration algorithm to reduce motion artifacts (Kraus M F et al., Biomed. Opt. Express. 2014; 5(8):2591-13). In addition, the projection resolved OCTA algorithm was applied to all OCTA scans to remove flow projection artifacts in the deeper layers (Zhang M et al., Biomed. Opt. Express. 2016; 7(3):816-28; Wang J et al., Biomed. Opt. Express. 2017; 8(3):1536-48). Scans with a signal strength index (SSI) lower than 50 were excluded. Table 1 shows various data characteristics for DR classification:

TABLE 1
Data for DR classification

| | Characteristics | | | | | | |
|---|---|---|---|---|---|---|---|
| | rDR classification | | vtDR classification | | Multiclass DR classification | | |
| Severity | nrDR | rDR | nvtDR | vtDR | nrDR | r/nvtDR | vtDR |
| Number of eyes | 199 | 257 | 280 | 176 | 199 | 81 | 176 |
| Age, mean | 48.8 | 58.4 | 52.2 | 57.5 | 48.8 | 60.4 | 57.5 |
| (SD), y | (14.6) | (12.1) | (14.7) | (12.3) | (14.6) | (14.7) | (12.3) |
| Female, % | 50.8% | 49.0% | 50.0% | 49.4% | 50.8% | 48.2% | 49.4% |

DR = diabetic retinopathy;
rDR = referable DR;
vtDR = vision threatening DR;
r/nvtDR = referable but not vision threatening DR

A masked trained retina specialist (TSH) graded 7-field color fundus photographs based on Early Treatment of Diabetic Retinopathy Study (ETDRS) scale (Ophthalmology. 1991; 98(5):823-33; Ophthalmoscopy D, Levels E. International clinical diabetic retinopathy disease severity scale detailed table. 2002). The presence of DME was determined using the central subfield thickness from structural OCT based on the Diabetic Retinopathy Clinical Research Network (DRCR.net) standard (Flaxel C J et al., Ophthalmology. 2020; 127(1):66-145). nrDR was defined as ETDRS level better than 35 and without DME; referable DR as ETDRS level 35 or worse, or any DR with DME; r/nvtDR as ETDRS levels 35-47 without DME; and vtDR as ETDRS level 53 or worse or any stage of DR with DME (Wong T Y et al., Ophthalmology, 2018; 125(10): 1608-22). The participants were enrolled after an informed consent in accordance with an Institutional Review Board approved protocol. The study complied with the Declaration of Helsinki and the Health Insurance Portability and Accountability Act.

### Data Inputs

Optical coherence tomography and OCTA generate detailed depth-resolved structural and microvascular information from the fundus. Extracting DR-related features using neural networks can, however, be more challenging and time consuming from 3D volumes such as those produced by OCTA than from 2D sources like fundus photography.

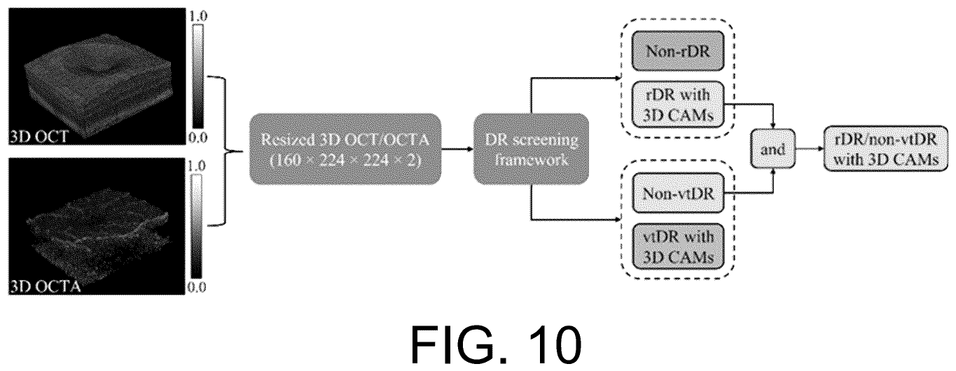

FIG. 10 illustrates an example automated DR classification framework using volumetric OCT and OCTA data as inputs. To improve the computational and space efficiency of the framework, each volumetric OCT and OCTA were resized to 160×224×224 voxels (a 160×224×224 structural and a 160×224×224 angiographic volume), and normalized to voxel values between 0 and 1. The input was the combination of each pair of resized volumes, giving final input dimensions of 160×224×224×2 pixels. These inputs were fed into a DR screening framework based on a 3D CNN architecture. The network produced two outputs: a non-referable (nrDR) or referable (rDR) DR classification, and a non-vision-threatening (nvtDR) or vision threatening (vtDR) DR classification. The multiclass DR classification result is defined based on the rDR and vtDR classification results. Class activation maps (CAMs) are also output for each classification result.

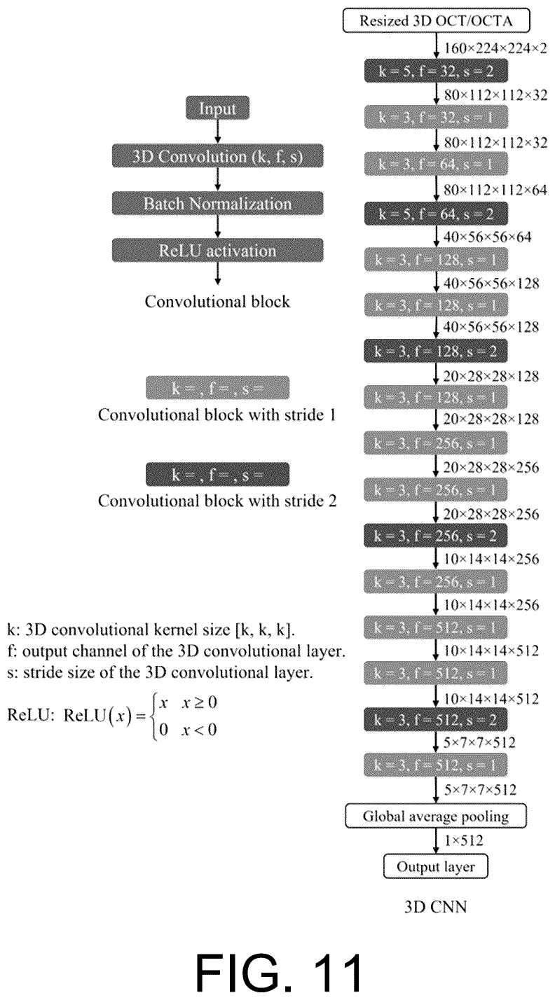

The novel 3D CNN architecture shown in FIG. 10, which includes 16 convolutional layers, was designed and used as the core classifier in the DR classification framework (FIG. 11). Five convolutional layers with stride 2 were used to downsample the input data. To avoid losing small but important DR-related features, diminishing convolutional kernel sizes were used in the five downsampling layers. Batch normalization (Ioffe S, Szegedy C. Batch normalization: accelerating deep network training by reducing internal covariate shift. arXiv preprint arXiv:1502.03167. 2015) was used after each 3D convolutional layer to increase convergence speed. In order to improve the computational efficiency while ensuring the resolution of the features, most of the 3D convolutional layers were used with the middle size inputs (after the first downsampling, but before the last). A global average pooling layer was used after the last 3D convolutional layer to generate the 1D input for the output layers.

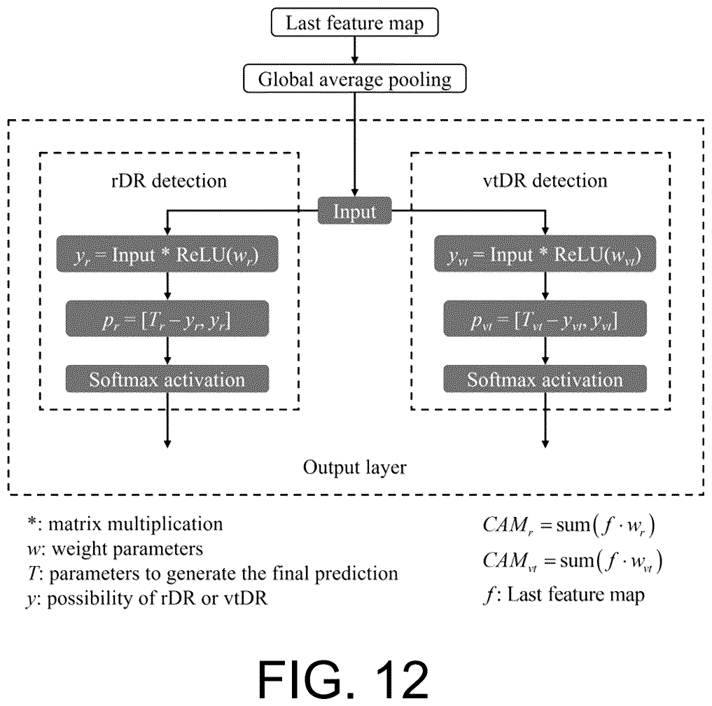

One subtlety in this example approach for multiclass classification is the need to correctly identify rDR/nvtDR eyes. Familiar frameworks for image classification like those used to diagnose medical conditions rely on the positive identification features associated with the malady. In the example framework, rDR and vtDR classification works similarly by using rectified linear unit (ReLU) activations in the last convolutional layer and weight parameters of all the fully connected layers to guarantee positive-definite prediction values (Nair V & Hinton G E, Proc. 27th ICML. 2010:807-14; Glorot X et al., Proc. 14th AISTATS. 2011:315-23). However, the identification of r/nvtDR does not depend on just the presence of rDR associated features, but also the absence of vtDR-associated features. To solve this issue, two parallel output layers were respectively used to detect rDR and vtDR at the same time (see FIG. 10). Each output layer was constructed by a fully connected layer with a softmax function (FIG. 12 in the Supplement). The inputs data can be then classified as nrDR, r/nvtDR, or vtDR based on rDR and vtDR classification outputs.

### Evaluation and Statistical Analysis

Overall accuracy, quadratic-weighted Cohen's kappa (Cohen J. A coefficient of agreement for nominal scales. Educational and psychological measurement. 1960; 20(1):37-46), and area under the receiver operating characteristic curve (AUC) were used to evaluate the DR classification performance of our framework. Among these evaluation metrics, the AUCs were used as the primary metrics for rDR and vtDR classifications. For the multiclass DR classification, the quadratic-weighted kappa was used as the primary metric. Five-fold cross-validation was used in each case to explore robustness. From the whole data set, 60%, 20%, and 20% of the data were split for training, validation, and testing, respectively. Care was taken to ensure data from the same patients were only included in one of either the training, validation, or testing data sets. The parameters and hyperparameters in the example framework were trained and optimized only using the training and validation data set. In addition, adaptive label smoothing was used during training to reduce the overfitting (Zang P et al., IEEE transactions on Biomedical Engineering. 2021; 68(6):1859-70).

### 3D Class Activation Maps (CAM) and Evaluation

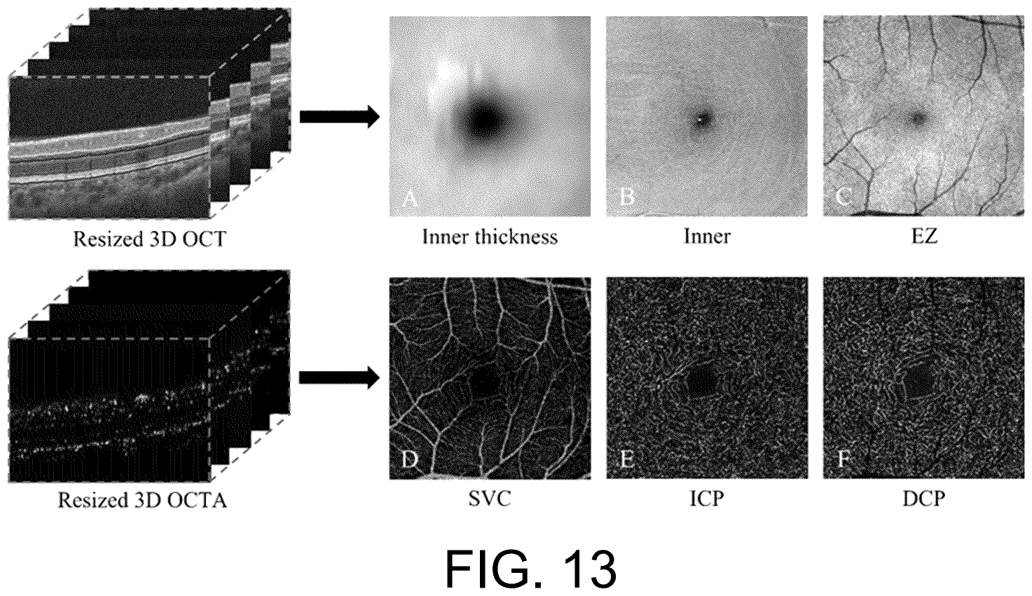

For the detected rDR and vtDR cases, the 3D CAMs were generated by projecting the weight parameters from corresponding output layer back to the feature maps of the last 3D convolutional layer before global average pooling (FIG. 12). For example, for each input, a CAM is the weighted sum of the last feature map (e.g., the 5×7×7×512 data set) before global average pooling based on the weight parameter (e.g., 1×512 data set) of one prediction layer, as illustrated in FIG. 12. After the weighted sum, the CAM is resized to the original size of the input (e.g., 160×224×224). To assess whether or not the framework correctly identified pathological regions, 3D CAMs were overlaid on en face or cross-sectional OCT and OCTA images. In order to generate the en face projections, an automated algorithm segmented the following retinal layers (FIG. 13): inner limiting membrane (ILM), nerve fiber layer (NFL), ganglion cell layer (GCL), inner plexiform layer (IPL), inner nuclear layer (INL), outer plexiform layer (OPL), outer nuclear layer (ONL), ellipsoid zone (EZ), retinal pigment epithelium (RPE), and Bruch's membrane (BM). For the cases with severe pathologies, trained graders manually corrected the layer segmentation when necessary, using custom software (Zhang M et al., Biomed. Opt. Express. 2015; 6(12):4661-75). From OCT volumes, the inner retinal (the slab between the Vitreous/ILM and OPL/ONL) thickness map was generated, en face mean projection of OCT reflectance, and EZ en face mean projection (ONL/EZ to EZ/RPE). From OCTA volumes, the superficial vascular complex (SVC), intermediate capillary plexus (ICP), and deep capillary plexus (DCP) angiograms (Zhang M et al., Investig. Ophthalmol. Vis. Sci. 2016; 57(13):5101-06; Campbell J P et al., Sci. Rep. 2017; 7:42201; Hormel T T et al., Biomed. Opt. Express. 2018; 9(12):6412-24) were generated. The SVC was defined as the inner 80% of the ganglion cell complex (GCC), which included structures between the ILM and IPL/INL border. The ICP was defined as the outer 20% of the GCC and the inner 50% of the INL. The DCP was defined as the remaining slab internal to the outer boundary of the OPL. The segmentation step and projection maps were used for evaluating the usefulness of 3D CAMs, not as input to the classification framework of this example.

### Results

TABLE 2
Automated DR classification performances

| | rDR | vtDR | Multiclass DR |
|---|---|---|---|
| Metric | classification | classification | classification |
| Overall | 91.52% ± 1.87% | 87.39% ± 2.02% | 81.52% ± 1.19% |
| accuracy | | | |
| Sensitivity | 90.77% ± 4.28% | 82.22% ± 2.83% | Not applicable |
| Specificity | 92.50% ± 3.16% | 90.71% ± 3.46% | Not applicable |
| AUC (mean ± | 0.96 ± 0.01 | 0.92 ± 0.02 | Not applicable |
| std) | | | |
| Quadratic- | 0.83 ± 0.04 | 0.73 ± 0.04 | 0.83 ± 0.03 |
| Weighted | | | |
| Kappa | | | |

DR = diabetic retinopathy; rDR = referable diabetic retinopathy; vtDR = vision threatening diabetic retinopathy; AUC = area under the receiver operating characteristic curve.

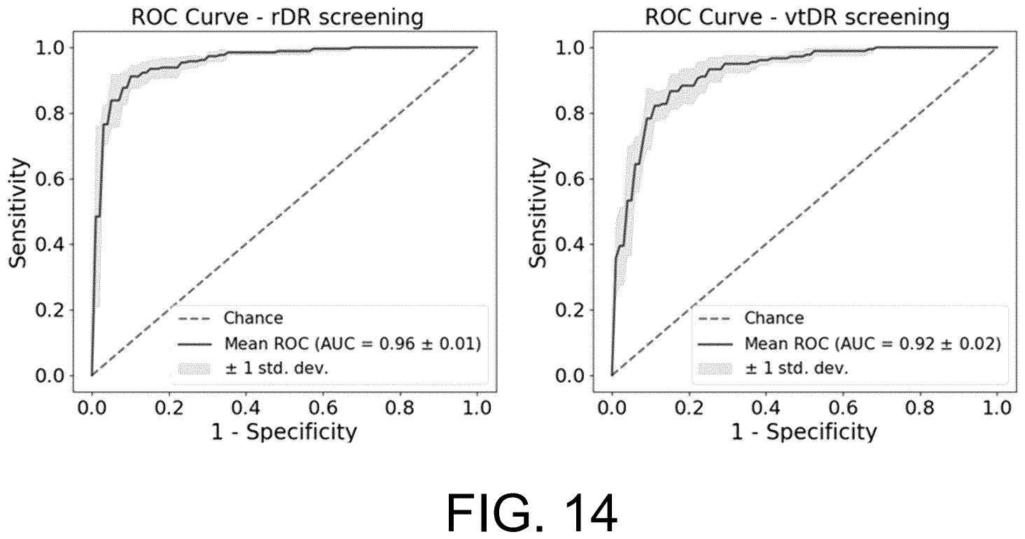

Model performance was the best for rDR classification, followed by vtDR, and multi-level DR classification (Table 2, FIG. 14). For the multiclass DR classification, which classifies each case as nrDR, r/nvtDR, or vtDR, we achieved a quadratic-weighted kappa 0.83, which is on par with the performance of ophthalmologists and retinal specialists (0.80 to 0.91) (Krause J et al., Ophthalmology. 2018; 125(8):1264-72). The network was notably better at classifying rDR and vtDR compared to r/nvtDR (Table 2). Most false positive r/nvtDR eyes were classified as vtDR (66.67%) instead of nrDR (33.33%).

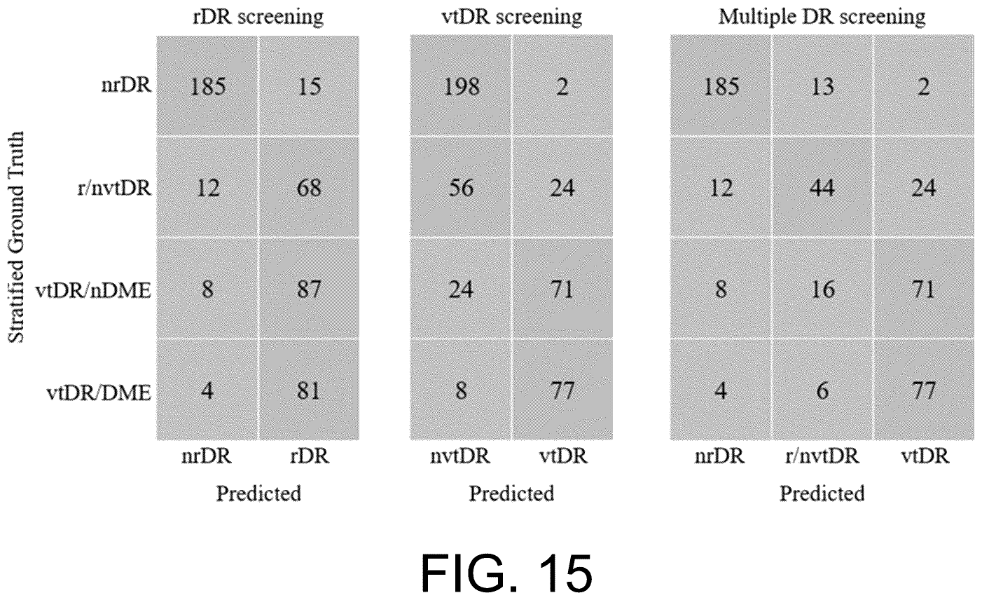

FIG. 15 illustrates three confusion matrices for referable DR (rDR) classification, vision threatening DR (vtDR) classification, and multiclass DR classification based on the overall 5-fold cross-validation results. The vtDR was split as non-DME (nDME) and DME in the matrices. The correctly and incorrectly classified cases are shaded blue and orange, respectively.

To demonstrate the deep-learning performance more explicitly, the stratified ground truth was compared with the network prediction with confusion matrices using the overall values from 5-fold cross-validation, as shown in FIG. 15. In the three confusion matrices, the vtDR cases were separated into non-DME (nDME) and DME to investigate whether the presence of DME can affect rDR and vtDR classification accuracy. In the rDR classification task, the classification accuracies of vtDR/nDME and vtDR/DME were found to be similar (87/95 and 81/85). For vtDR classification, the network identified cases with DME (77/85) with a greater accuracy than nDME cases (71/95), which may imply DME features were likely influential for decision making. In the multi-level classification, the network misclassified 16/95 vtDR/nDME cases as r/nvtDR. In addition, most of the r/nvtDR cases with false-positive results were classified as vtDR. Only 2 nrDR cases were misidentified as vtDR.

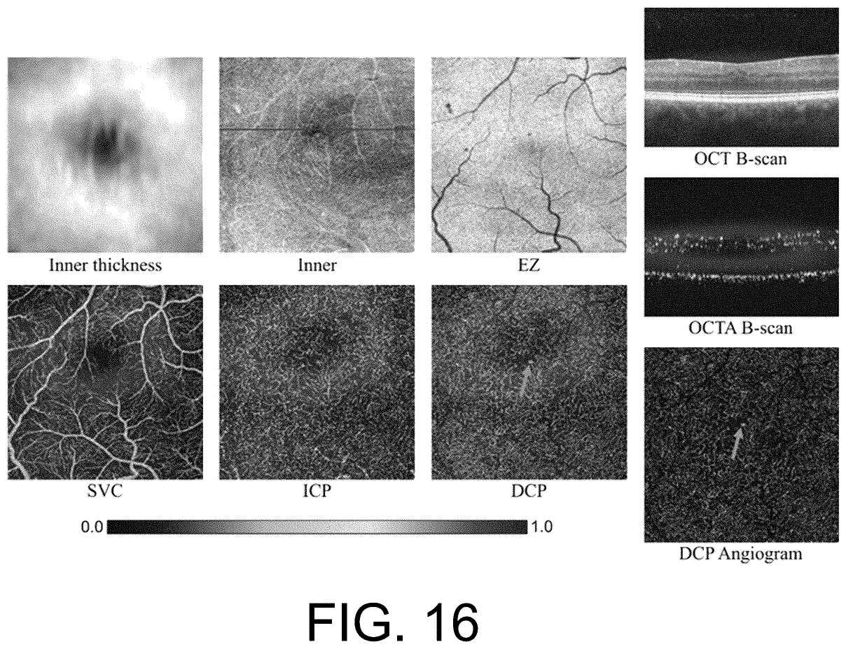

FIG. 16 illustrates class activation maps (CAMs) based on the referable DR (rDR) output layer of the example framework for data from an eye with rDR without vision threatening DR (vtDR). Six en face projections covered with the corresponding projections of the 3D CAMs are shown. Extracted CAMs for an OCT and OCTA B-scans (red line in the inner retina en face projection) are also shown. The deep capillary plexus (DCP) angiogram without a CAM is shown so that the pathology highlighted by the corresponding CAM can be more easily identified. The green arrows indicate an abnormal vessel in the DCP. The en face projections shown are the inner retinal (the slab between the Vitreous/inner limiting membrane and outer plexiform and outer nuclear layer boundaries) thickness map mean projection of the OCT reflectance, ellipsoid zone (EZ) en face mean projection (Outer nuclear layer/ellipsoid zone boundary to ellipsoid zone/retinal pigment epithelium boundary), and maximum projection of the flow volume in the superficial vascular complex (SVC; inner 80% of the ganglion cell complex), intermediate capillary plexus (ICP; outer 20% of the ganglion cell complex and inner 50% of the inner nuclear layer), and deep capillary plexus (DCP; remaining slab internal to the outer boundary of the outer plexiform layer).

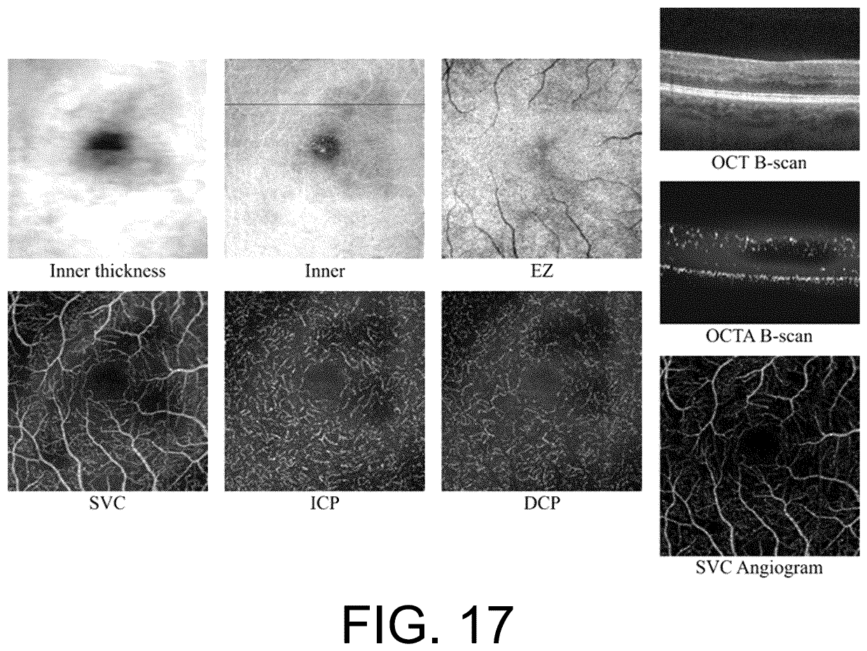

FIG. 17 illustrates class activation maps (CAMs) based on the vision threatening DR (vtDR) output layer of the example framework for data from an eye with vtDR but without DME. Six en face projections covered with corresponding projections of the 3D CAMs are shown. Extracted CAMs for an OCT and OCTA B-scan (red line in the inner retina en face projection) are also shown. An SVC angiogram without a CAM is also shown to help identify pathological features for comparison. The SVC CAM indicates that the framework learned to identify non-perfusion areas, which are known biomarkers for DR diagnosis.

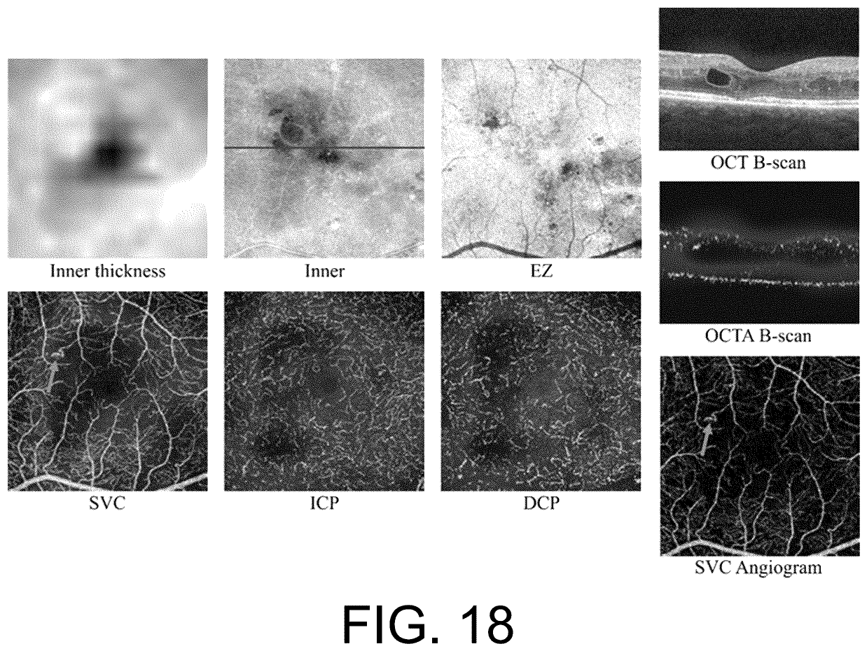

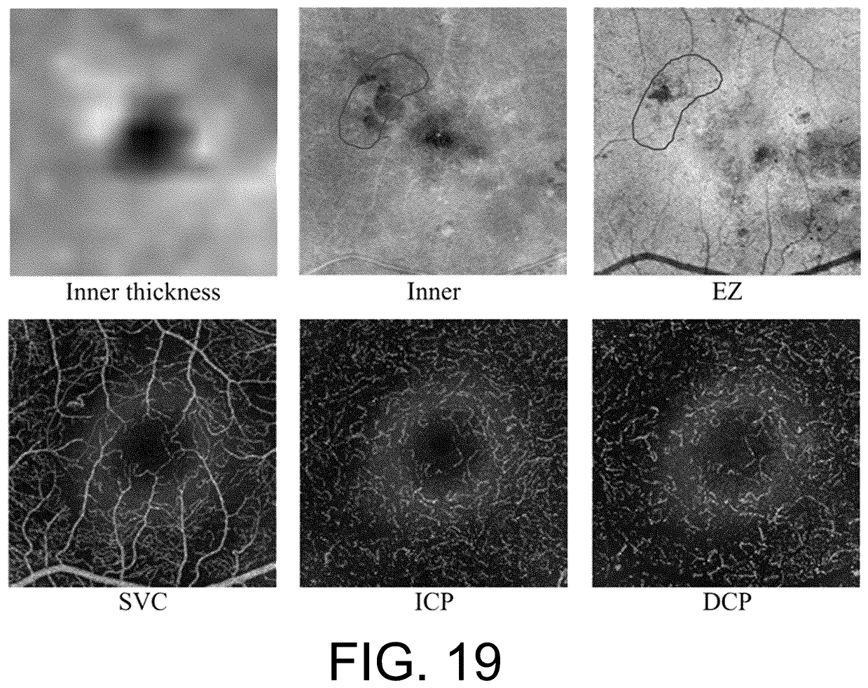

To better understand network decision making, CAMs were produced for some example cases. The CAM output of a r/nvtDR case points to dilated vessels in the DCP and a perifoveal area of decreased vessel density (FIG. 16). Meanwhile, in a vtDR case without DME, the CAMs have a larger area of high attention (FIG. 17), indicating that the DR pathology is more pervasive throughout the volume. In addition to pointing to areas of decreased vessel density, the CAM overlaid on a structural OCT B-scan points to an area with abnormal curvature of the retinal layers. Finally, for a vtDR case with DME, the CAM pointed to areas with intraretinal cysts and abnormal curvature of the retinal layers on structural OCT, as well as decreased vessel density and abnormally dilated vessels on OCTA (FIG. 18). This is an improvement over a previous 2D CAM output (FIG. 19) (Zang P et al., IEEE transactions on Biomedical Engineering. 2021; 68(6):1859-70), which identified changes in the perifoveal region, but missed other pathologies, such as intraretinal cysts and abnormally dilated vessels.

FIG. 18 illustrates class activation maps (CAMs) based on vision threatening DR (vtDR) output layer of our framework for data from an eye with vtDR and DME. Six en face projections covered with the corresponding projections of 3D CAMs are shown. Extracted CAMs for an OCT and OCTA B-scan (red line in the inner retina en face projection) are also shown. The SVC angiogram without a CAM is shown to more readily observe pathology. The green arrow in the SVC CAM shows an abnormal vessel, which can also be seen in the angiogram. Central macular fluid is marked by green circle on the OCT B-scan. The CAM allocated high weights to both of these regions. For descriptions of the regions projected over to produce the en face images, see the description of FIG. 16.

### Discussion

In this study, a CNN-based automated DR classification framework that operates directly on volumetric OCT/OCTA data without requiring retinal layer segmentation was analyzed. This framework classified cases into clinically actionable categories (nrDR, r/nvtDR, and vtDR) using a single imaging modality. For multiclass DR classification, the framework achieved a quadratic-weighted kappa of 0.83±0.03, which is on par with the performance of human ophthalmologists and retinal specialists (0.80 to 0.91) (Krause J et al., Ophthalmology. 2018; 125(8):1264-72). The network also demonstrated robust performance on both rDR and vtDR classification (AUC=0.96±0.01; 0.92±0.02, respectively). These results indicate that the example framework achieved automated DR classification using only OCT/OCTA at a specialist-level of performance for DR classification.

The framework used feature-rich structural OCT and OCTA volumes as inputs and a deep-learning model as the core classifier to achieve a high level of performance. The majority of DR classification algorithms to date have been based on fundus photographs (Gargeya R & Leng T, Ophthalmology. 2017; 124(7):962-69; Abramoff M D et al., Investig. Ophthalmol. Vis. Sci. 2016; 57(13):5200-06; Gulshan V, et al., JAMA. 2016; 316(22):2402-10; Ghosh R et al., Proc. 4th SPIN. 2017:550-54). However, fundus photographs detect DME with only about a 70% accuracy relative to structural OCT, while DME accounts for the majority of vision loss in DR (Lee R et al., Eye and vision. 2015; 2(1):1-25; Prescott G et al., Brit. J. Ophthalmol. 2014; 98(8):1042-49). The example method described herein, on the other hand, actually performs better in the presence of DME (see Table 2).

The image labels appealed to structural OCT to detect DME, and so did not adhere exactly to the ETDRS scale (the current gold standard for DR grading), which uses only seven field fundus photographs. This prevented the model from learning to misdiagnose eyes based on the presence of DME not detected by fundus photography. However, at the same time OCTA may not recapitulate every feature in fundus photography used for staging DR on the ETDRS scale. For example, OCTA does not detect intraretinal hemorrhages and may not detect all microaneurysms (Jia Y et al., Opt. Express. 2012; 20(4):4710-25). Achieving comparable performance to fundus photographs-based automated classification frameworks indicates that these disadvantages were surmounted by our approach.

Another important feature in the example framework design is the use of a deep-learning model for the classifier. Compared to previous OCT/OCTA-based DR classification algorithms utilizing 2D en face projection images as inputs, and which only classified rDR, the example framework described herein has at least several innovations. One advantage is the use of the whole 3D volume, instead of pre-selected features from segmented en face images. This means that correlations or structures within the data volume that may be difficult for a human to identify can still be incorporated into decision making. 2D approaches may miss important features without access to cross-sectional information, as happens with color fundus photography and DME (Le D et al., Transl. Vis. Sci. Technol. 2020; 9(2):35). As a corollary, the example framework may then also have a greater capacity to improve with more training data since no data is removed by projection. The addition of a volume scan provides much more information that can be learned than the addition of a single image. Moreover, accurate retinal layer segmentation is required to generate the en face images. In severely diseased eyes, automated layer segmentations often fail. Mis-segmented layers can introduce artifacts into en face images unless they are manually corrected, a labor-intensive task that may not be clinically practical. By using volumetric data, the example framework avoids this issue entirely. Another advantage built into the example framework is the ability to detect both rDR and vtDR. This higher level of granularity makes a more efficient use of resources possible compared to solutions that only identify rDR (Gargeya R & Leng T, Ophthalmology. 2017; 124(7):962-69; Gulshan V, et al., JAMA. 2016; 316(22):2402-10; Sandhu H S et al., Investig. Ophthalmol. Vis. Sci. 2018; 59(7):3155-60; Sandhu H S et al., Brit. J. Ophthalmol. 2018; 102(11):1564-69; Alam M et al., Retina. 2020; 40(2):322-32; Heisler M et al., Transl. Vis. Sci. Technol. 2020; 9(2):20; Le D et al., Transl. Vis. Sci. Technol. 2020; 9(2):35).

A final significant advantage in the example framework is the inclusion of CAMs. While independent of model performance, generating CAMs allow clinicians to interpret the classification results and ensure model outputs are correct. This is important since, outside of visualizations such as CAMs, users cannot in general ascertain how deep learning algorithms arrive at a classification decision. However, in medical imaging it is essential to be able to verify and understand these classification decisions since doing so could prevent misdiagnosis. Black-box algorithms such as deep learning algorithms may hide important biases that could prove to be disadvantageous for certain groups. This risk can be lowered when the results are interpretable. With the example framework described herein, this is possible. Previous CAM generation approaches are not suitable for automated DR classification since classifying an eye into nrDR or nvtDR should not depend on the presence of features, but rather the absence. Therefore, the example framework used ReLU (Nair V & Hinton G E, Proc. 27th ICML. 2010:807-14; Glorot X et al., Proc. 14th AISTATS. 2011:315-23) activations on all the variables and weight parameters in the output layers to force the CNN and CAMs to only detect and highlight unique features belong to rDR or vtDR. The CAMs in this work were generated volumetrically. Compared to 2D CAMs, the current framework using 3D OCT/OCTA as inputs can identify and learn relevant features (FIG. 18 and FIG. 19). The resulting CAMs consistently highlighted macular fluid (FIG. 18), demonstrating that the model did indeed learn relevant features since central macular fluid is the most important biomarker for detecting DME (You Q S et al., JAMA Ophthalmol. 2021; 139(7):734-41). The 3D CAMs also were found to point to other key features such as lower vessel density and dilated capillaries (FIGS. 3 and 4). Although the 3D CAM did not identify all DR features (e.g., certain regions with lower vessel density were ignored), it found many key features, indicating that the example framework has successfully learned relevant features and that 3D CAMs could be useful in clinical review. In addition, the purpose of generating 3D CAMs is not necessarily to find all DR biomarkers, but simply to highlight the features used by the network to make decisions. That the network ignored some known DR-associated features is interesting, since it implies that these features were not critical for diagnosing DR at a given severity.

### Conclusion

This example proposes a fully automated DR classification framework using 3D OCT and OCTA as inputs. The example framework achieved reliable performance on multiclass DR classification (nrDR, rDR/nvtDR, and vtDR), and produces 3D CAMs that can be used to interpret the model's decision making. By using the example framework, the number of imaging modalities required for DR classification was reduced from fundus photographs and OCT to an OCTA procedure alone. This accuracy of the model output in this study also suggests the combination of OCT/OCTA and deep learning could perform well in a clinical setting.

### EXAMPLE CLAUSES

The following clauses provide various implementations of the present disclosure.
1. A medical device for diabetic retinopathy (DR) identification, the medical device including: an optical coherence tomography (OCT) scanner configured to obtain a three-dimensional (3D) image of a retina, the 3D image including voxels, an example voxel among the voxels including a first value representing an OCT value of an example volume and a second value representing an OCTA value of the example volume; at least one processor; and memory storing instructions that, when executed by the at least one processor, cause the at least one processor to perform operations including: generating, by a convolutional neural network (CNN) and using the 3D image, a vector; generating, by a first model and using the vector, a first likelihood that the retina exhibits a first level of DR; generating, by a second model and using the vector, a second likelihood that the retina exhibits a second level of DR; and determining whether the retina exhibits an absence of DR, the first level of DR, or the second level of DR based on the first likelihood and the second likelihood; and a display configured to output an indication of whether the retina exhibits the absence of DR, the first level of DR, or the second level of DR.
2. The medical device of clause 1, wherein generating, by the CNN and using the 3D image, the vector includes: processing, by multiple convolution blocks arranged in parallel, the 3D image, and wherein the multiple convolution blocks include at least one first convolution block with a stride of 1 and at least one second convolution block with a stride of 2.
3. The medical device of clause 1 or 2, wherein generating, by the first model and using the vector, the first likelihood includes: generating a first intermediary matrix by multiplying the vector by a ReLU of weight parameters associated with the first level of DR; generating a first probability matrix based on the first intermediary matrix and first parameters; and generating the first likelihood by performing softmax activation on the first probability matrix, and wherein generating, by the second model and using the vector, the second likelihood includes: generating a second intermediary matrix by multiplying the vector by a ReLU of weight parameters associated with the second level of DR; generating a second probability matrix based on the second intermediary matrix and second parameters; and generating the second likelihood by performing softmax activation on the second probability matrix.
4. The medical device of any one of clauses 1 to 3, wherein the operations further include: generating, based on the 3D image, a CAM indicating at least one region in the 3D image that is indicative of DR, and wherein the display is further configured to output the CAM.
5. The medical device of any one of clauses 1 to 4, wherein the operations further include: training the CNN based on training data.
6. A method, including: identifying a 3D image of a retina; generating, by a CNN and using the 3D image, a vector; generating, by a first model and using the vector, a first likelihood that the retina exhibits a first level of an ophthalmic disease; generating, by a second model and using the vector, a second likelihood that the retina exhibits a second level of the ophthalmic disease; determining whether the retina exhibits an absence of the ophthalmic disease, the first level of the ophthalmic disease, or the second level of the ophthalmic disease based on the first likelihood and the second likelihood; and outputting an indication of whether the retina exhibits the absence of the ophthalmic disease, the first level of the ophthalmic disease, or the second level of the ophthalmic disease.
7. The method of clause 6, wherein identifying the 3D image of the retina includes simultaneously performing an OCT scan and an OCTA scan on the retina.
8. The method of clause 6 or 7, wherein the 3D image includes voxels, an example voxel among the voxels including a first value associated with an OCTA value of a volume of the retina and a second value associated with an OCT value of the volume.
9. The method of any one of clauses 6 to 8, wherein generating, by the CNN and using the 3D image, the vector includes: processing, by multiple convolution blocks arranged in parallel, the 3D image, and wherein the multiple convolution blocks include at least one first convolution block with a stride of 1 and at least one second convolution block with a stride of 2.
10. The method of clause 9, wherein an example convolution block among the multiple convolution blocks includes a 3D convolution layer, a batch normalization layer, and a ReLU activation layer.
11. The method of any one of clauses 6 to 10, wherein generating, by the first model and using the vector, the first likelihood that the retina exhibits the first level of an ophthalmic disease includes: generating a first intermediary matrix by multiplying the vector by a ReLU of weight parameters associated with the first level of the ophthalmic disease; generating a first probability matrix based on the first intermediary matrix and first parameters; and generating the first likelihood by performing softmax activation on the first probability matrix.
12. The method of any one of clauses 6 to 11, wherein generating, by the second model and using the vector, the second likelihood that the retina exhibits the second level of an ophthalmic disease includes: generating a second intermediary matrix by multiplying the vector by a ReLU of weight parameters associated with the second level of the ophthalmic disease; generating a second probability matrix based on the second intermediary matrix and second parameters; and generating the second likelihood by performing softmax activation on the second probability matrix.
13. The method of any one of clauses 6 to 12, wherein determining whether the retina exhibits an absence of the ophthalmic disease, the first level of the ophthalmic disease, or the second level of the ophthalmic disease based on the first likelihood and the second likelihood includes: comparing the first likelihood and the second likelihood.
14. The method of any one of clauses 6 to 13, wherein determining whether the retina exhibits an absence of the ophthalmic disease, the first level of the ophthalmic disease, or the second level of the ophthalmic disease based on the first likelihood and the second likelihood includes: comparing the first likelihood to a first threshold and/or comparing the second likelihood to a second threshold.
15. The method of any one of clauses 6 to 14, wherein outputting the indication includes causing a display to visually output the indication.
16. The method of any one of clauses 6 to 15, wherein outputting the indication includes transmitting, to an external computing device, a signal including the indication.
17. The method of any one of clauses 6 to 16, further including: generating a CAM based on the vector, the CAM indicating one or more regions of the retina including features associated with the ophthalmic disease; and visually outputting the CAM.
18. A system, including: at least one processor; and memory storing instructions that, when executed by the at least one processor, cause the at least one processor to perform operations including the method of one of clauses 6 to 17.
19. The system of clause 18, further including: an OCT and/or OCTA device configured to generate the 3D image of the retina by performing an OCT and/or OCTA scan on the retina.
20. A non-transitory computer-readable medium encoding instructions to perform the method of one of clauses 6 to 17.

### Conclusion

The environments and individual elements described herein may of course include many other logical, programmatic, and physical components, of which those shown in the accompanying figures are merely examples that are related to the discussion herein.

Other architectures may be used to implement the described functionality and are intended to be within the scope of this disclosure. Furthermore, although specific distributions of responsibilities are defined above for purposes of discussion, the various functions and responsibilities might be distributed and divided in different ways, depending on circumstances.

Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

As will be understood by one of ordinary skill in the art, each embodiment disclosed herein can comprise, consist essentially of, or consist of its particular stated element(s), step(s), ingredient(s), and/or component(s). Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiments.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Explicit definitions and explanations used in the present disclosure are meant and intended to be controlling in any future construction unless clearly and unambiguously modified in examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A medical device for diabetic retinopathy (DR) identification, the medical device comprising:
an optical coherence tomography (OCT) scanner configured to obtain a three-dimensional (3D) image of a retina, the 3D image comprising voxels, an example voxel among the voxels comprising a first value representing an OCT value of an example volume and a second value representing an OCTA value of the example volume;
at least one processor; and
memory storing instructions that, when executed by the at least one processor, cause the at least one processor to perform operations comprising:
generating, by a convolutional neural network (CNN) and using the 3D image, a vector;
generating, by a first model and using the vector, a first likelihood that the retina exhibits a first level of DR;
generating, by a second model and using the vector, a second likelihood that the retina exhibits a second level of DR; and
determining whether the retina exhibits an absence of DR, the first level of DR, or the second level of DR based on the first likelihood and the second likelihood; and
a display configured to output an indication of whether the retina exhibits the absence of DR, the first level of DR, or the second level of DR.

2. The medical device of claim 1, wherein generating, by the CNN and using the 3D image, the vector comprises:
processing, by multiple convolution blocks arranged in parallel, the 3D image, and
wherein the multiple convolution blocks comprise at least one first convolution block with a stride of 1 and at least one second convolution block with a stride of 2.

3. The medical device of claim 1, wherein generating, by the first model and using the vector, the first likelihood comprises:
generating a first intermediary matrix by multiplying the vector by a ReLU of weight parameters associated with the first level of DR;
generating a first probability matrix based on the first intermediary matrix and first parameters; and
generating the first likelihood by performing softmax activation on the first probability matrix, and
wherein generating, by the second model and using the vector, the second likelihood comprises:
generating a second intermediary matrix by multiplying the vector by a ReLU of weight parameters associated with the second level of DR;
generating a second probability matrix based on the second intermediary matrix and second parameters; and
generating the second likelihood by performing softmax activation on the second probability matrix.

4. The medical device of claim 1, wherein the operations further comprise:
generating, based on the 3D image, a CAM indicating at least one region in the 3D image that is indicative of DR, and
wherein the display is further configured to output the CAM.

5. The medical device of claim 1, wherein the operations further comprise:
training the CNN based on training data.

6. A method, comprising:
identifying a 3D image of a retina;
generating, by a CNN and using the 3D image, a vector;
generating, by a first model and using the vector, a first likelihood that the retina exhibits a first level of an ophthalmic disease;
generating, by a second model and using the vector, a second likelihood that the retina exhibits a second level of the ophthalmic disease;
determining whether the retina exhibits an absence of the ophthalmic disease, the first level of the ophthalmic disease, or the second level of the ophthalmic disease based on the first likelihood and the second likelihood; and
outputting an indication of whether the retina exhibits the absence of the ophthalmic disease, the first level of the ophthalmic disease, or the second level of the ophthalmic disease.

7. The method of claim 6, wherein identifying the 3D image of the retina comprises simultaneously performing an OCT scan and an OCTA scan on the retina.

8. The method of claim 6, wherein the 3D image comprises voxels, an example voxel among the voxels comprising a first value associated with an OCTA value of a volume of the retina and a second value associated with an OCT value of the volume.

9. The method of claim 6, wherein generating, by the CNN and using the 3D image, the vector comprises:
processing, by multiple convolution blocks arranged in parallel, the 3D image, and
wherein the multiple convolution blocks comprise at least one first convolution block with a stride of 1 and at least one second convolution block with a stride of 2

10. The method of claim 9, wherein an example convolution block among the multiple convolution blocks comprises a 3D convolution layer, a batch normalization layer, and a ReLU activation layer.

11. The method of claim 6, wherein generating, by the first model and using the vector, the first likelihood that the retina exhibits the first level of an ophthalmic disease comprises:
generating a first intermediary matrix by multiplying the vector by a ReLU of weight parameters associated with the first level of the ophthalmic disease;
generating a first probability matrix based on the first intermediary matrix and first parameters; and
generating the first likelihood by performing softmax activation on the first probability matrix.

12. The method of claim 6, wherein generating, by the second model and using the vector, the second likelihood that the retina exhibits the second level of an ophthalmic disease comprises:
generating a second intermediary matrix by multiplying the vector by a ReLU of weight parameters associated with the second level of the ophthalmic disease;
generating a second probability matrix based on the second intermediary matrix and second parameters; and
generating the second likelihood by performing softmax activation on the second probability matrix.

13. The method of claim 6, wherein determining whether the retina exhibits an absence of the ophthalmic disease, the first level of the ophthalmic disease, or the second level of the ophthalmic disease based on the first likelihood and the second likelihood comprises:
comparing the first likelihood and the second likelihood.

14. The method of claim 6, wherein determining whether the retina exhibits an absence of the ophthalmic disease, the first level of the ophthalmic disease, or the second level of the ophthalmic disease based on the first likelihood and the second likelihood comprises:
comparing the first likelihood to a first threshold and/or comparing the second likelihood to a second threshold.

15. The method of claim 6, wherein outputting the indication comprises causing a display to visually output the indication.

16. The method of claim 6, wherein outputting the indication comprises transmitting, to an external computing device, a signal comprising the indication.

17. The method of claim 6, further comprising:
generating a CAM based on the vector, the CAM indicating one or more regions of the retina comprising features associated with the ophthalmic disease; and
visually outputting the CAM.

18. A system, comprising:
at least one processor; and
memory storing instructions that, when executed by the at least one processor, cause the at least one processor to perform operations comprising:
identifying a 3D image of a retina;
generating, by a CNN and using the 3D image, a vector;
generating, by a first model and using the vector, a first likelihood that the retina exhibits a first level of an ophthalmic disease;
generating, by a second model and using the vector, a second likelihood that the retina exhibits a second level of the ophthalmic disease;
determining whether the retina exhibits an absence of the ophthalmic disease, the first level of the ophthalmic disease, or the second level of the ophthalmic disease based on the first likelihood and the second likelihood; and
outputting an indication of whether the retina exhibits the absence of the ophthalmic disease, the first level of the ophthalmic disease, or the second level of the ophthalmic disease.

19. The system of claim 18, further comprising:
an OCT and/or OCTA device configured to generate the 3D image of the retina by performing an OCT and/or OCTA scan on the retina.

20. The system of claim 18, further comprising:
a transceiver,
wherein the processor is configured to output the indication of whether the retina exhibits the absence of the ophthalmic disease, the first level of the ophthalmic disease, or the second level of the ophthalmic disease by causing the transceiver to transmit, to an external device, one or more data packets comprising the indication.
